(19) United States
(12) Patent Application Publication
(10) Pub. No.: US 2023/0094393 A1
(43) Pub. Date: Mar. 30, 2023

(54) PEPTIDE PARTICLES AND METHODS OF FORMATION

(71) Applicant: Elektrofi, Inc., Boston, MA (US)

(72) Inventors: Lyndon Fitzgerald Charles, Medford, MA (US); Chase Spenser Coffman, Newton, MA (US); Daniel Benjamin Dadon, East Boston, MA (US); Chaitanya Sudrik, Stoneham, MA (US)

(21) Appl. No.: 17/759,594

(22) PCT Filed: Feb. 5, 2021
(86) PCT No.: PCT/US2021/016878

§ 371 Date: Jul. 27, 2022

Related U.S. Application Data
(60) Provisional application No. 62/971,685, filed on Feb. 7, 2020.

(51) Int. Cl. A61K 9/16; A61K 38/03; A61K 41/17

(52) U.S. Cl. CPC A61K 9/1694; A61K 9/1623; A61K 9/1617; A61K 38/03; A61K 41/17

(57) ABSTRACT

The present disclosure relates to compositions and methods that enable the formation of pharmaceutically relevant particles that can be used for therapy. In particular, the methods disclosed herein allow the controlled formation of circular particles comprising biologically active peptides.

### RELATED APPLICATION(S)

This application claims the benefit of U.S. Provisional Application No. 62/971,685, filed Feb. 7, 2020.

The entire teachings of the above application are incorporated herein by reference.

### TECHNICAL FIELD

The present disclosure relates to compositions and methods that enable the formation of biologically active peptide particles that can be used for therapy. In particular, the methods disclosed herein allow the formation of circular particles comprising biologically active peptides.

### BACKGROUND

Materials science and the application of nanotechnology calls for more efficient, reproducible and innovative technologies to synthesize novel functional particles. Recent advances in synthesis and the controlled assembly of bioactive particles have enabled their applications for use in therapy. Current efforts have been directed to developing new synthetic approaches for non-circular microparticles that often exhibit physical properties unobtainable by simply tuning the size and form of the particles. However, the application of these techniques to circular particles have been limited due to the lack of sufficient control over size uniformity, shape selectivity, surface functionality and skeletal density of the particles which are often difficult to obtain. Therefore, a highly robust and controlled method for circular particle preparation is needed.

### SUMMARY

Provided herein is a particle or a composition comprising a plurality of particles, comprising a peptide, wherein the particle comprises up to about 10% (w/w) of peptide and the circularity of the particle is from about 0.10 to about 1.00.

In one aspect, the disclosure provides a particle comprising a peptide, wherein the particle comprises up to about 10% (w/w) of peptide and the circularity of the particle is from about 0.10 to about 1.00.

In another aspect, the disclosure provides a composition comprising a plurality of particles comprising a peptide suspended in a liquid, wherein the particles comprise up to about 10% (w/w) of peptide and the circularity of the particle is from about 0.10 to about 1.00.

The present disclosure also provides a method of forming particles.

In one aspect, the disclosure provides a method of forming particles, the method comprising:

a) providing droplets comprising a first liquid and a peptide;

b) contacting the droplets comprising the peptide with a second liquid;

c) allowing the droplets to dry; and

d) removing the first and second liquids,

thereby forming particles comprising a peptide, wherein the particles comprise up to about 10% (w/w) of peptide and the circularity of the particles is from about 0.10 to about 1.00 after removing the first and second liquids.

The present compositions and methods may be useful for the formation of pharmaceutically relevant peptide particles that can be used for therapy. In preferred embodiments, the methods disclosed herein may allow the formation of circular particles comprising a peptide.

### BRIEF DESCRIPTION OF THE DRAWINGS

The foregoing will be apparent from the following more particular description of example embodiments, as illustrated in the accompanying drawings in which like reference characters, refer to the same parts throughout the different views. The drawings are not necessarily to scale, emphasis instead being placed upon illustrating embodiments.

FIG. 1 shows an image of peptide particles at 20 μm produced using vortex to form droplets of the disclosure.

FIG. 2 shows an image of peptide particles at 20 μm produced using an ultrasonic atomizer to form droplets of the disclosure.

### DETAILED DESCRIPTION

Particles have been produced using various techniques. For example, the generation of particles can be accomplished by producing a droplet of a liquid comprising a peptide dissolved in a solvent. The solvent can then be extracted from the droplets by depositing the droplets into a liquid in which the solvent, but not the peptide, is soluble leaving behind a solid particle. Isolation of the particles occur following removal of the liquids. However, the application of these techniques to form functional circular particles have been limited due to the lack of sufficient control over size uniformity, shape selectivity, surface functionality and skeletal density of the particles which are often difficult to obtain. The present disclosure seeks to mitigate the control issues that are associated with forming functional particles by providing a robust and controlled method for particle preparation.

The present disclosure generally relates to a particle comprising a peptide or a composition comprising a plurality of particles comprising a peptide suspended in a liquid, wherein the particle or the plurality of particles comprises up to about 10% (w/w) of peptide and the circularity of the particle is from about 0.10 to about 1.00.

The present disclosure also relates to methods of forming particles, the method comprising: a) providing droplets comprising a first liquid and a peptide; b) contacting the droplets comprising the peptide with a second liquid; c) allowing the droplets to dry; and d) removing the first and second liquids, thereby forming particles comprising a peptide, wherein the particles comprise up to about 10% (w/w) of peptide and the circularity of the particles is from about 0.10 to about 1.00 after removing the first and second liquids.

As described herein, the disclosure provides methods for the preparation of particles including one or more biologically active peptides. The particles can be formed by creating droplets of a first liquid that includes a biologically active peptide, and removing the first liquid through its dispersal in a second liquid to solidify the droplets. The process of forming the particles as described herein, significantly alters the structure or morphology of the particles and may enhance the stability of the peptides. For example, the biologically active particles may be stored for extended periods of time without significant loss of activity or the need for refrigeration. These particles may be used to generate stabilized pharmaceutical compositions, pharmaceutical suspension formulations, pharmaceutical powder formulations (e.g., inhalable powders, injectable powders), creams or other topical pastes, nutraceuticals, or cosmetics. The term “pharmaceutical composition” as used herein, denotes a composition in which a peptide retains, or partially retains, its intended biological activity or functional form, and in which only pharmaceutically acceptable components are included.

It will be readily understood that the aspects and embodiments, as generally described herein, are exemplary. The following more detailed description of various aspects and embodiments are not intended to limit the scope of the present disclosure, but is merely representative of various aspects and embodiments. Moreover, the compositions and methods disclosed herein may be changed by those skilled in the art without departing from the scope of the present disclosure. Unless defined otherwise, all technical and scientific terms used herein have the same meaning as is commonly understood by one of skill in the art to which this disclosure belongs. All publications and patents referred to herein are incorporated by reference.

### Definitions

For purposes of the present disclosure, the following definitions will be used unless expressly stated otherwise:

The terms “a”, “an”, “the” and similar referents used in the context of describing the present disclosure are to be construed to cover both the singular and the plural, unless otherwise indicated herein or clearly contradicted by context. All methods described herein, can be performed in any suitable order unless otherwise indicated herein or otherwise clearly contradicted by context. The use of any and all examples, or exemplary language (e.g., “such as”) provided herein, is intended merely to better illuminate the present disclosure and does not pose a limitation on the scope of the disclosure otherwise claimed. No language in the present specification should be construed as indicating any unclaimed element is essential to the practice of the disclosure.

The term “about” in relation to a given numerical value, such as for temperature and period of time, is meant to include numerical values within 10% of the specified value.

As used herein, an “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, iso-butyl, sec-butyl, tert-butyl, n-pentyl, tert-pentyl, neo-pentyl, iso-pentyl, sec-pentyl, 3-pentyl, sec-iso-pentyl, active-pentyl, hexyl, heptyl, octyl, ethylhexyl, and the like. A C₁₋₈ straight chained or branched alkyl group is also referred to as a “lower alkyl” group. An alkyl group with two open valences is sometimes referred to as an alkylene group, such as methylene, ethylene, propylene and the like. Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, an alkyl, a halogen, a hydroxyl, a carbonyl (such as a carboxyl, and alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamide, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN and the like. In other embodiments, the term “alkyl” can mean “cycloalkyl” which refers to a non-aromatic carbocyclic ring having 3 to 10 carbon ring atoms, which are carbon atoms bound together to form the ring. The ring may be saturated or have one or more carbon-carbon double bonds. Examples of cycloalkyl include, but not limited to cyclopropyl, cyclobutyl, cyclopentyl, cyclopentenyl, cyclohexyl, cyclohexenyl, and cycloheptyl, as well as bridged and caged saturated ring groups such as norbornyl and adamantyl. As described herein, organic solvents include, but are not limited to aliphatic hydrocarbon solvents, aromatic hydrocarbon solvents, alcohols or alkylalcohols, alkylethers, sulfoxides, alkylketones, alkylacetates, trialkylamines, alkylformates, trialkylamines, or a combination thereof. Aliphatic hydrocarbon solvents can be pentane, hexane, heptane, octane, cyclohexane, and the like or a combination thereof. Aromatic hydrocarbon solvents can be benzene, toluene, and the like or a combination thereof. Alcohols or alkylalcohols include, for example, methanol, ethanol, propanol, butanol, pentanol, hexanol, heptanol, octanol, decanol, amylalcohol, or a combination thereof. Alkylethers include methyl, ethyl, propyl, butyl, and the like, e.g., diethylether, diisopropylether or a combination thereof. Sulfoxides include dimethyl sulfoxide (DMSO), decylmethyl sulfoxide, tetradecylmethyl sulfoxide, and the like or a combination thereof. The term “alkylketone” refers to a ketone substituted with an alkyl group, e.g., acetone, ethylmethylketone, and the like or a combination thereof. The term “alkylacetate” refers to an acetate substituted with an alkyl group, e.g., ethylacetate, propylacetate (n-propylacetate, iso-propylacetate), butylacetate (n-butylacetate, iso-butylacetate, sec-butylacetate, tert-butylacetate), amylacetate (n-pentylacetate, tert-pentylacetate, neo-pentylacetate, iso-pentylacetate, sec-pentylacetate, 3-pentylacetate, sec-iso-pentylacetate, active-pentylacetate), 2-ethylhexylacetate, and the like or a combination thereof. The term “alkylformate” refers to a formate substituted with an alkyl group, e.g., methylformate, ethylformate, propylformate, butylformate, and the like or a combination thereof. The term “trialkylamine” refers to an amino group substituted with three alkyl groups, e.g., triethylamine.

As used herein, an “amino acid” or “residue” refers to any naturally or non-naturally occurring amino acid, any amino acid derivative or any amino acid mimic known in the art. Included are the L- as well as the D-forms of the respective amino acids, although the L-forms are usually preferred. In some embodiments, the term relates to any one of the 20 naturally occurring amino acids: glycine (Gly), alanine (Ala), valine (Val), leucine (Leu), isoleucine (Ile), proline (Pro), cysteine (Cys), methionine (Met), serine (Ser), threonine (Thr), glutamine (Gin), asparagine (Asn), glutamic acid (Glu), aspartic acid (Asp), lysine (Lys), histidine (His), arginine (Arg), phenylalanine (Phe), tryptophan (Trp), and tyrosine (Tyr) in their L-form. In certain embodiments, the amino acid side-chain may be a side-chain of Gly, Ala, Val, Leu, Ile, Met, Cys, Ser, Thr, Trp, Phe, Lys, Arg, His, Tyr, Asn, Gln, Asp, Glu, or Pro. Abbreviations used herein to identify specific amino acids may use the three or one-letter code as known in the art.

As used herein, except where the context requires otherwise, the term “comprise” and variations of the term, such as “comprising”, “comprises” and “comprised”, are not intended to exclude further additives, components, integers or steps. The terms “including” and “comprising” may be used interchangeably. As used herein, the phrases “selected from the group consisting of”, “chosen from”, and the like, include mixtures of the specified materials. Where a numerical limit or range is stated herein, the endpoints are included. Also, all values and subranges within a numerical limit or range are specifically included as if explicitly written out. References to an element in the singular is not intended to mean “one and only one” unless specifically stated, but rather “one or more”. Unless specifically stated otherwise, terms such as “some” refer to one or more, and singular terms such as “a”, “an” and “the” refer to one or more.

The term “oligopeptide” is used to refer to a peptide with fewer members of amino acids as opposed to a polypeptide or protein. Oligopeptides described herein, are typically comprised of about two to about forty amino acid residues. Oligopeptides include dipeptides (two amino acids), tripeptides (three amino acids), tetrapeptides (four amino acids), pentapeptides (five amino acids), hexapeptides (six amino acids), heptapeptides (seven amino acids), octapeptides (eight amino acids), nonapeptides (nine amino acids), decapeptides (ten amino acids), undecapeptides (eleven amino acids), dodecapeptides (twelve amino acids), icosapeptides (twenty amino acids), tricontapeptides (thirty amino acids), tetracontapeptides (forty amino acids), etc. Oligopeptides may also be classified according to molecular structure: aeruginosins, cyanopeptolins, microcystins, microviridins, microginins, anabaenopeptins and cyclamides, etc. Homo-oligopeptides are oligopeptides comprising the same amino acid. In preferred embodiments, homo-oligopeptides comprise 10 amino acid poly-valine, poly-alanine, and poly-glycine hexamers.

The meaning of the term “peptides” are defined as small proteins of two or more amino acids linked by the carboxyl group of one to the amino group of another. Accordingly, at its basic level, peptide synthesis of whatever type comprises the repeated steps of adding amino acid or peptide molecules to one another or to an existing peptide chain. The term “peptide” generally has from about 2 to about 100 amino acids, whereas a polypeptide or protein has about 100 or more amino acids, up to a full length sequence which may be translated from a gene. Additionally, as used herein, a peptide can be a subsequence or a portion of a polypeptide or protein. In certain embodiments, the peptide consists of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues. In preferred embodiments, the peptide is from about 2 to about 12 amino acids in length. In some embodiments, the peptide is from about 2 to about 20 amino acids in length.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the term “prodrug” is intended to encompass peptides which, under physiologic conditions, are converted into the therapeutically active peptides of the present disclosure. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present disclosure. In certain embodiments, some or all of the molecules in a composition represented above can be replaced with the corresponding suitable prodrug, e.g., wherein a hydroxyl in the parent molecule is presented as an ester or a carbonate or carboxylic acid present in the peptide is presented as an ester.

The meaning of the term “protein” is defined as a linear polymer built from about 20 different amino acids. The type and the sequence of amino acids in a protein are specified by the DNA that produces them. In certain embodiments, the sequences can be natural and unnatural. The sequence of amino acids determines the overall structure and function of a protein. In some embodiments, proteins can contain 50 or more residues. In preferred embodiments, proteins can contain greater than about 101 residues in length. A protein's net charge can be determined by two factors: 1) the total count of acidic amino acids vs. basic amino acids; and 2) the specific solvent pH surroundings, which expose positive or negative residues. As used herein, “net positively or net negatively charged proteins” are proteins that, under non-denaturing pH surroundings, have a net positive or net negative electric charge. In general, those skilled in the art will recognize that all proteins may be considered “net negatively charged proteins,” regardless of their amino acid composition, depending on their pH and/or solvent surroundings. For example, different solvents can expose negative or positive side chains depending on the solvent pH. Proteins or peptides are preferably selected from any type of enzyme or antibodies or fragments thereof showing substantially the same activity as the corresponding enzyme or antibody. Proteins or peptides may serve as a structural material (e.g. keratin), as enzymes, as hormones, as transporters (e.g. hemoglobin), as antibodies, or as regulators of gene expression. Proteins or peptides are required for the structure, function, and regulation of cells, tissues, and organs.

The term “substantially” as used herein, refers to a majority of, or mostly, as in at least about 50%, 60%, 70%, 80%, 90%, 95%, 96%, 97%, 98%, 99%, 99.5%, 99.9%, 99.99%, or at least about 99.999% or more.

It is understood that the specific order or hierarchy of steps in the methods or processes disclosed is an illustration of exemplary approaches. Based upon design preferences, it is understood that the specific order or hierarchy of steps in the methods or processes may be rearranged. Some of the steps may be performed simultaneously. The accompanying methods claims present elements of the various steps in a sample order, and are not meant to be limited to a specific hierarchy or order presented. A phrase such as “embodiment” does not imply that such embodiment applies to all configurations of the subject technology. A disclosure relating to an embodiment may apply to all embodiments, or one or more embodiments. A phrase such as an embodiment may refer to one or more embodiments and vice-versa.

### Particles

Unless otherwise defined, all terms of art, notations and other scientific terminology used herein, are intended to have the meanings commonly understood by those of skill in the art to which this disclosure pertains. In some cases, terms with commonly understood meanings are defined herein, for clarity and/or for ready reference, and the inclusion of such definitions herein, should not necessarily be construed to represent a substantial difference over what is generally understood in the art. The techniques and procedures described or referenced herein, are generally well understood and commonly employed using conventional methodology by those skilled in the art. As appropriate, procedures involving the use of commercially available kits and reagents are generally carried out in accordance with manufacturer defined protocols and/or parameters unless otherwise noted. As used herein, the phrase “and/or” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

In some aspects, the disclosure relates to a particle comprising a peptide, wherein the particle comprises up to about 10% (w/w) of peptide and the circularity of the particle is from about 0.10 to about 1.00.

The terms “particle” or “particles” or “microparticle” or “microparticles” are used herein, interchangeably in the broadest sense, refers to a discrete body or bodies. The particles described herein, are circular, spheroidal and of controlled dispersity with a characteristic size from sub-micrometers to tens of micrometers, in contrast to, e.g., a porous monolithic “cake”, which is typically produced during conventional lyophilization. This morphology allows for a flowable powder (as described by low Hausner ratios) without post-processing. In some embodiments, the term “particle” refers to a quantity of a peptide or peptides which is either in a state of matter that is substantially solid as compared to a liquid droplet or in a gel form. In other embodiments, the particle may include a core and a shell, where the shell may be viewed as an encapsulant. In still other embodiments, the particle does not include a shell, in which case, the particle is made up entirely of a core. The term “proto-particle” refers to a stage of particle formation in which one or more of the components comprising the particle are in an at least a partial state of desiccation. The total liquid content of the proto-particle is less than that of the droplet and greater than that of the formed particle. Similarly, the average concentration of the solutes is higher than that of the drop but typically less than that of the formed particle. The term “encapsulant” refers to a substance that can be dried or gelled around a particle core to form a shell.

As disclosed herein, the peptide is a biologically active peptide. The term “biologically active” refers to the ability of a peptide described herein to effect a biological change in a subject, modulate a biological receptor or mechanism in a subject, or have a physiological effect in a subject. In some embodiments, the biologically active peptide comprises from about 2 to about 12 amino acid residues. The biologically active peptide in the particles may have an activity per unit of about 0.5 to about 1.0, about 0.75 to about 1.0 activity per unit, or about 0.9 to about 1.0 activity per unit. Activity is measured relative to the same biologically active peptide prior to particle formation. In certain embodiments, the biologically active peptide has an activity per unit of about 0.5 to about 1.0. In preferred embodiments, the peptide has an activity per unit of about 0.5 to about 1.0. The term “activity” refers to the ratio of a functional or structural aspect of a peptide at two points in time. The denominator of the ratio corresponds to a measure of the functional or structural aspect of the peptide in the feed solution, immediately in advance of droplet formation. The numerator of the ratio corresponds to the same measure of a functional or structural aspect of the peptide at a later point in time, e.g., immediately after particle formation.

In some embodiments, the particle comprises up to about 10% (w/w), e.g., from about 9% (w/w), about 8% (w/w), about 7% (w/w), about 6% (w/w), about 5% (w/w), about 4% (w/w), about 3% (w/w), about 2% (w/w), or about 1% (w/w), of peptide. In other embodiments, the particle comprises up to about 8% (w/w) of peptide. In still other embodiments, the particle comprises up to about 6% (w/w) of peptide. In certain other embodiments, the particle comprises up to about 5% (w/w) of peptide. In certain embodiments, the particle comprises up to about 3% (w/w) of peptide.

The particles according to the disclosure are circular. Circularity can serve as an indicator of the shape of the particle. The particles described herein, can have a characteristic circularity, e.g., have a relative shape, that is substantially circular. This characteristic describes and defines the form of a particle on the basis of its circularity. The circularity is 1.0 when the particle has a completely circular structure. Particles as described herein, can have a circularity greater than about 0.80, e.g., of about 0.90, 0.95, 0.96, 0.97, 0.98, or about 0.99. In some embodiments, the circularity of the particle is greater than about 0.80. In other embodiments, the circularity of the particle is greater than about 0.85. In certain embodiments, the circularity of the particle is greater than about 0.90. In certain preferred embodiments, the circularity of the particle is greater than about 0.95. In preferred embodiments, the circularity of the particle is greater than about 0.98. The diameter and the circularity of the particles can be determined by the image processing of an image observed under an electron microscope or the like or a flow-type particle image analyzer. The circularity can also be determined by subjecting particles to circularity measurement and averaging the resulting values. For example, circularity (circ) can be calculated using the following formula:

$$circ = 4 * π * \frac{Area}{{Perimeter}^{2}} . \qquad Eq . 1$$

The term “perimeter”, as used herein, refers to the boundary of a closed plane figure or the sum of all borders of a two-dimensional image. As used herein, the term “area”, refers to the crossectional area of a two-dimensional image of a particle. The circularity of a particle can also be described as the ratio of the smallest diameter of the particle to its largest diameter. For a perfect circle, the ratio is 1. The percentage circularity can be calculated by multiplying the circularity by 100. The circularity can be calculated, for example, by measuring the aspect ratio using any software adapted to deal with images, for example, images obtained by microscopy, in particular, scanning electron microscopy (SEM) or transmission electron microscopy (TEM).

In other embodiments, the circularity of the particle is from about 0.10 to about 1.00, e.g., from about 0.20, 0.30, 0.40, 0.50. 0.60, 0.70, 0.75, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99 to about 1.00. In certain embodiments, the circularity of the particle is from about 0.80 to about 1.00. In still other embodiments, the circularity of the particle is from about 0.85 to about 1.00. In certain other embodiments, the circularity of the particle is from about 0.90 to about 1.00. In certain preferred embodiments, the circularity of the particle is from about 0.95 to about 1.00. In preferred embodiments, the circularity of the particle is from about 0.98 to about 1.00. In other embodiments of the disclosure, the circularity of the particles is about 1.00. In some embodiments, methods of measuring particle circularity include image analysis of scanning electron micrographs of the particles in which the average roundness is calculated on the basis of the cross-sectional shapes of the particles projected onto the plane of the image. Such roundness factors can be extended to identify the corresponding circularity.

In some embodiments of the disclosure, the drying operation may be controlled to provide particles having particular characteristics, such as particles having a substantially smooth surface. “Surface roughness”, as used herein, means a particle having numerous wrinkles or creases, e.g., being ridged or wrinkled. The term “pit”, as used herein, refers to an indentation or crevice in the particle, either an indentation or crevice in the two-dimensional image or an indentation or crevice in an object. The term “spike”, as used herein, refers to a projection pointing outward from the centroid of a particle, a projection pointing outward from the centroid of a two-dimensional image or a sharp projection pointing outward from an object.

In preferred embodiments of the disclosure, the particles as described herein, have a surface morphology that is smooth rather than ridged or wrinkled. The surface roughness of the particles may be decreased by controlling the formulation and/or process to form the particles as described herein. In certain embodiments, the drying conditions can be selected to control the particle morphology in order to enhance the smoothness of the particle's surface. In particular, the drying conditions can be selected to provide particles having a substantially smooth surface. In preferred embodiments, the particle has a substantially smooth surface. A person of ordinary skill in the field of this disclosure can readily assess the surface morphology of the disclosed particles using routine and standard techniques.

The particle as described herein, is intentionally controlled in its diameter. In some embodiments, the particle has a diameter of about 0.1 to about 1000 μm, e.g., from about 0.1 to about 900, 800, 700, 600, 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, or about 0.2 μm. In other embodiments, the particle has a diameter of about 1 to about 100 μm, e.g., from about 2, 3, 4, 5, 6, 7, 8, 9, 10, 11, 12, 13, 14, 15, 16, 17, 18, 19, 20, 25, 30, 35, 40, 45, or 50 to about 100 μm. In still other embodiments, the particle has a diameter of about 0.1 to about 100 μm. In certain other embodiments, the particle has a diameter of about 1 to about 100 μm. In certain embodiments, the particle has a diameter of about 5 to about 100 μm. In certain preferred embodiments, the particle has a diameter of about 5 to about 50 μm. In preferred embodiments, the particle has a diameter of about 5 to about 20 μm.

In other embodiments, the particle exhibits a skeletal density of about 1.00 to about 6.00 g/cm³, e.g., from about 1.00 to about 5.00 g/cm³, from about 1.00 to about 3.00 g/cm³, from about 1.00 to about 2.00 g/cm³, from about 1.15 to about 1.60 g/cm³, from about 1.25 to about 1.50 g/cm³, from about 1.30 to about 1.40 g/cm³, or from about 1.32 to about 1.40 g/cm³. In some embodiments, the particle has a skeletal density of about 1.00 to about 6.00 g/cm³. In still other embodiments, the particle has a skeletal density of about 1.15 to about 1.60 g/cm³. In certain other embodiments, the particle has a skeletal density of about 1.25 to about 1.50 g/cm³. In preferred embodiments, the particle has a skeletal density of about 1.30 to about 1.40 g/cm³. Exemplary methods of skeletal density measurements include gas displacement pycnometry.

In certain embodiments, the particle has a skeletal density of about 1000 mg/mL to about 1500 mg/mL, about 1050 mg/mL to about 1500 mg/mL, about 1100 mg/mL to about 1500 mg/mL, about 1150 mg/mL to about 1500 mg/mL, about 1200 mg/mL to about 1500 mg/mL, about 1250 mg/mL to about 1500 mg/mL, about 1300 mg/mL to about 1500 mg/mL, about 1310 mg/mL to about 1500 mg/mL, about 1320 mg/mL to about 1500 mg/mL, about 1330 mg/mL to about 1500 mg/mL, about 1340 mg/mL to about 1500 mg/mL, about 1350 mg/mL to about 1500 mg/mL, about 1360 mg/mL to about 1500 mg/mL, about 1370 mg/mL to about 1500 mg/mL, about 1380 mg/mL to about 1500 mg/mL, about 1390 mg/mL to about 1500 mg/mL, about 1400 mg/mL to about 1500 mg/mL, about 1410 mg/mL to about 1500 mg/mL, about 1420 mg/mL to about 1500 mg/mL, about 1430 mg/mL to about 1500 mg/mL, about 1440 mg/mL to about 1500 mg/mL, about 1450 mg/mL to about 1500 mg/mL, about 1460 mg/mL to about 1500 mg/mL, about 1470 mg/mL to about 1500 mg/mL, about 1480 mg/mL to about 1500 mg/mL, or about 1490 mg/mL to about 1500 mg/mL.

In some embodiments, the particle can be characterized by a glass transition temperature of about 0° C. to about 250° C., e.g., of about 34° C. to about 200° C., of about 50° C. to about 200° C., of about 50° C. to about 200° C., of about 60 to about 170° C., of about 60to about 130° C., of about 60 to about 100° C., or of about 60 to about 90° C. The term “glass transition” as used herein, refers to a thermodynamic transition of an amorphous material characterized by step changes in specific heat capacity and modulus. At temperatures above the glass transition temperature, molecular mobility is increased as are the rates of physical and chemical changes. Exemplary analytical methods for the determination of the glass transition temperature include differential scanning calorimetry and dynamic mobility analysis. In other embodiments, the particle has a glass transition temperature that is higher than about 60° C. In still other embodiments, the particle has a glass transition temperature that is higher than about 90° C. In certain other embodiments, the particle has a glass transition temperature that is higher than about 100° C. In certain preferred embodiments, the particle has a glass transition temperature that is higher than about 130° C. In preferred embodiments, the particle has a glass transition temperature that is higher than about 170° C. In certain preferred embodiments, the particles are heated to about ±30° C., e.g., to about ±20, ±10, ±5, ±1° C., of the glass transition temperature of the particles during drying.

In other embodiments, the particle further comprises a carbohydrate, a protein stabilizer, an emulsifier, an amino acid, a surfactant, or a combination thereof.

In some embodiments, the carbohydrate may be from the families of monosaccharides, disaccharides, oligosaccharides, or polysaccharides. In other embodiments, the carbohydrate is dextran, trehalose, sucrose, agarose, mannitol, lactose, sorbitol, maltose, starch, alginates, xanthan, galactomanin, agar, agarose, or a combination thereof. In certain embodiments, the carbohydrate is dextran, trehalose, sucrose, agarose, mannitol, lactose, sorbitol, maltose, or a combination thereof. In preferred embodiments, the carbohydrate is trehalose, cyclodextrins, hydroxypropyl beta-cyclodextrin, sulfobutylether beta-cyclodextrin, or a combination thereof. Cyclodextrins are available in three different forms a, (3, and y based on the number of number of glucose monomers. The number of glucose monomers in a, (3, and y cyclodextrin can be 6, 7, or 8, respectively.

In other embodiments, the protein stabilizer is acetyltryptophanate, caprylate, N-acetyltryptophan, trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, or a combination thereof. In certain embodiments, the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethyl starch, N-methyl-2-pyrrolidone, sorbitol, sucrose, mannitol, cyclodextrin, saccharides, hydroxypropyl beta-cyclodextrin, sulfobutylether beta-cyclodextrin, or a combination thereof. In preferred embodiments, the protein stabilizer is trehalose, cyclodextrin, hydroxypropyl beta-cyclodextrin, sulfobutylether beta-cyclodextrin, or a combination thereof. In certain preferred embodiments, the PEG is PEG 200, PEG 300, PEG 3350, PEG 8000, PEG 10000, PEG 20000, or a combination thereof. The stabilizers, used synonymously with the term “stabilizing agent”, as described herein, can be a salt, a carbohydrate, saccharides or amino acids, preferably a carbohydrate or saccharide admitted by the authorities as a suitable additive or excipient in pharmaceutical compositions. The term “excipient” refers to an additive to a preparation or formulation, which may be useful in achieving a desired modification to the characteristics of the preparation or formulation. Such modifications include, but are not limited to, physical stability, chemical stability, and therapeutic efficacy. Exemplary excipients include, but are not limited to a carbohydrate, a pH adjusting agent, a salt, a chelator, a mineral, a polymer, a surfactant, an amino acid, an oligopeptide, a biologic excipient, a chemical excipient, an antiseptic, an antioxidant, a paraben, a bactericide, a fungicide, a vitamin, a preservative, an analgesic, and/or nutrient media.

Examples of emulsifiers suitable for use in the particle include, but are not limited to, lipophilic agents having an HLB of less than 7, such as mixed fatty acid monoglycerides; mixed fatty acid diglycerides; mixtures of fatty acid mono- and diglycerides; lipophilic polyglycerol esters; glycerol esters including glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; and mixtures thereof glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; or a combination thereof. In some embodiments, the emulsifier is polysorbate 80, polysorbate 60, polysorbate 20, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof. In preferred embodiments, the fatty acid ester of sorbitol is a sorbitan ester, e.g., span 20, span 40, span 60, or span 80. In certain preferred embodiments, the emulsifier is polysorbate 80, sorbitan monooleate, or a combination thereof.

In certain embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or various salts thereof (arginine hydrochloride, arginine glutamate, etc.) or a combination thereof. In preferred embodiments, the amino acid is arginine, histidine, proline, asparagine, or a combination thereof.

In some embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, sodium laureth sulfate, lecithin, or a combination thereof. In other embodiments, the surfactant includes, but is not limited to: (i) cationic surfactants such as; cetyltrimethylammonium chloride, hexadecyltrimethylammonium chloride, benzalkonium chloride, benzethonium chloride, dioctadecyldimethylammonium bromide; (ii) anionic surfactants such as magnesium stearate, sodium dodecyl sulfate, dioctyl sodium sulfosuccinate, sodium myreth sulfate, perfluorooctanesulfonate, alkyl ether phosphates; (iii) non-ionic surfactants such as alkylphenol ethoxylates (TritonX-100), fatty alcohol ethoxylates (octaethylene glycol monododecyl ether, cocamide diethanolamine, poloxamers, glycerolmonostearate, fatty acid esters of sorbitol (sorbitan monolaurate, Tween 80, Tween 20; and (iv) zwitterionic surfactants such as cocamidopropyl hydroxysultaine, and 3-[(3-Cholamidopropyl)dimethylammonio]-1-propanesulfonate (CHAPS). In certain embodiments, the surfactant is polysorbate, docusate or lecithin. In preferred embodiments, the surfactant is polysorbate 20, polysorbate 60, or polysorbate 80. In certain preferred embodiments, the surfactant is polysorbate 20 or polysorbate 80. In still other embodiments, the fatty acid ester of sorbitol is a sorbitan ester, e.g., span 20, span 40, span 60, or span 80. In certain other embodiments, the surfactant is an ionic surfactant.

In other embodiments, the particle has a surfactant content of less than about 10% by mass, e.g., less than about 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001% by mass. In some embodiments, the particle has a surfactant content of less than about 5% by mass. In certain embodiments, the particle has a surfactant content of less than about 3% by mass. In still other embodiments, the particle has a surfactant content of less than about 0.1% by mass. In certain other embodiments, the particle has a surfactant content of less than about 0.01% by mass. In some embodiments, the particle has a surfactant content of less than about 0.001% by mass. In preferred embodiments, the particle has a surfactant content of less than about 1% by mass. In certain preferred embodiments, the particle is substantially free from any surfactant content. Exemplary methods of measuring the surfactant content include reconstitution of the particles in an appropriate medium, e.g., deionized water, and subsequent analysis of the reconstituted solution through liquid chromatography. The chromatographic technique may include the use of a charged aerosol detector (CAD) or an evaporative light scattering detector (ELSD).

In some embodiments, the residual moisture or solvent content of the dry particle is less than about 7% by weight, e.g., less than about 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% by weight. In other embodiments, the particle has less than about 7% residual moisture by weight. In still other embodiments, the particle has less than about 5% residual moisture by weight. In certain embodiments, the particle has less than about 3% residual moisture by weight. In preferred embodiments, the particle has less than about 1% residual moisture by weight.

In other embodiments, the particle has about 1% to about 7% residual moisture by weight. In still other embodiments, the particle has about 1% to about 5% residual moisture by weight. In certain embodiments, the particle has about 1% to about 3% residual moisture by weight. In preferred embodiments, the particle is substantially free from any residual moisture by weight. Exemplary methods for the measurement of moisture content include chemical titration methods, e.g., Karl Fischer titration involving an oven. A variety of solvents, including water, may also be measured using weight loss methods involving thermal excitation. Exemplary methods include Thermogravimetric Analysis with Infrared Spectroscopy (TGA-IR) or Gas Chromatography Flame Ionization Detector Mass Spectrometry (GC-FID/MS).

The particle comprising at least one peptide described herein, can be prepared in a number of ways, as well as any methods of forming the particle disclosed in, for example, PCT/US2017/063150, PCT/US2018/043774, PCT/US2019/033875, PCT/US20/15957, and PCT/US20/050508, each of which is hereby incorporated by reference in its entirety.

While each of the elements of the present disclosure is described herein, as containing multiple embodiments, it should be understood that, unless indicated otherwise, each of the embodiments of a given element of the present disclosure is capable of being used with each of the embodiments of the other elements of the present disclosure and each such use is intended to form a distinct embodiment of the present disclosure.

It will be understood by one of ordinary skill in the relevant arts that other suitable modifications and adaptations to the compositions and methods described herein are readily apparent from the description of the disclosure contained herein, in view of information known to the ordinarily skilled artisan, and may be made without departing from the scope of the disclosure or any embodiment thereof.

### Pharmaceutical Compositions

In certain embodiments, the disclosure relates to a composition comprising a plurality of particles comprising any one of the aforementioned peptides suspended in a low viscosity liquid. In certain preferred embodiments, the disclosure relates to a pharmaceutical composition comprising a plurality of particles comprising any one of the aforementioned biologically active peptides suspended in a low viscosity pharmaceutically acceptable liquid.

The phrase “pharmaceutically acceptable” is employed herein, to refer to those biologically active peptides, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The term “pharmaceutically acceptable” can refer to particles and compositions comprising a plurality of particles that do not produce an adverse, allergic, or other untoward reaction when administered to a mammal, such as a human, as appropriate. The preparation of a pharmaceutical composition comprising an antibody or additional active ingredient will be known to those of skill in the art in light of the present disclosure. Moreover, for mammal (e.g., human) administration, it will be understood that preparations should meet sterility, pyrogenicity, general safety, and purity standards as required by FDA Office of Biological Standards.

The phrase “pharmaceutically acceptable liquid” includes any and all aqueous solvents (e.g., water, alcoholic/aqueous solutions, saline solutions, parenteral vehicles, such as sodium chloride, Ringer's dextrose, etc.), non-aqueous or organic solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters. In certain preferred embodiments, the particles are suspended in a pharmaceutically acceptable liquid. In preferred embodiments, the liquid is a pharmaceutically acceptable liquid.

A pharmaceutical composition (formulation) as described herein, can be administered to a subject by any of a number of routes of administration including, for example, parenterally (including intramuscularly, intravenously, subcutaneously or intrathecally as, for example, a sterile solution or suspension); intraperitoneally; or subcutaneously. In certain embodiments, a composition may be simply suspended in a non-aqueous or an organic liquid carrier. Details of appropriate routes of administration and compositions suitable for same can be found in, for example, U.S. Pat. Nos. 6,110,973; 5,763,493; 5,731,000; 5,541,231; 5,427,798; 5,358,970 and 4,172,896, as well as in patents cited therein. The term “suspension formulation” refers to a liquid formulation including solid particles disposed within a carrier liquid in which they are not soluble on an appropriate timescale. The particles may settle over time, i.e., the physical stability of the suspension is not indefinite, but may be re-suspended using a form of agitation or excitation.

A “therapeutic amount” refers to an amount of a biologically active peptide required to produce the desired effect. As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

In preferred embodiments according to the disclosure as described herein, the composition comprising a plurality of particles has improved stability of the biologically active peptide compared to an aqueous composition comprising the peptide in monomeric form.

In other aspects, the disclosure relates to a composition comprising a plurality of particles comprising a peptide suspended in a liquid, wherein the particles comprise up to about 10% (w/w) of peptide and the circularity of the particles are from about 0.10 to about 1.00. In certain embodiments, the peptide is a biologically active peptide. In preferred embodiments, the biologically active peptide comprises from about 2 to about 12 amino acid residues.

In some embodiments, the disclosure provides a composition containing a plurality of particles that includes a peptide where the storage stability of the peptide in the particles is improved with respect to the storage stability of the peptide in the first liquid. In other embodiments, storage conditions are defined by time (e.g., more than about 2 years, more than about 1 year, more than about 6 months, more than about 3 months, more than about 1 month, or more than about 1 week) and temperature (e.g., about −80° C. to about 100° C., about −80° C. to about 60° C., about −20° C. to about 60° C., about 4 to about 60° C.), among potentially other variables. In still other embodiments, the storage time is about 3 days, about 7 days, about 30 days, about 90 days, about 180 days, about 1 year, or about 2 years. In certain other embodiments, this temperature is about −80° C., about −40° C., about −20° C., about 4° C., about 25° C., about 40° C., or about 40 to about 60° C. In preferred embodiments, the storage stability of the peptide in the particles is improved with respect to the storage stability of a first liquid of the peptide.

In other embodiments, the particles comprise up to about 8% (w/w) of peptide. In still other embodiments, the particles comprise up to about 6% (w/w) of peptide. In certain other embodiments, the particles comprise up to about 5% (w/w) of peptide. In certain embodiments, the particles comprise up to about 3% (w/w) of peptide.

In some embodiments, the circularity of the particles is from about 0.80 to about 1.00. In other embodiments, the circularity of the particles is from about 0.85 to about 1.00. In still other embodiments, the circularity of the particles is from about 0.90 to about 1.00. In certain other embodiments, the circularity of the particles is from about 0.95 to about 1.00. In certain preferred embodiments, the circularity of the particles is from about 0.98 to about 1.00. In preferred embodiments, the circularity of the particles is about 1.00.

In preferred embodiments, the particles have a substantially smooth surface. A person of ordinary skill in the field of this disclosure can readily assess the surface morphology of the disclosed particles using routine and standard techniques.

In other embodiments, the particles have a diameter of about 0.1 to about 100 μm. In some embodiments, the particles have a diameter of about 1 to about 100 μm. In still other embodiments, the particles have a diameter of about 5 to about 100 μm. In certain other embodiments, the particles have a diameter of about 5 to about 50 μm. In certain embodiments, the particles have a diameter of about 5 to about 20 μm.

In some embodiments, the particles have a skeletal density of about 1.00 to about 6.00 g/cm³. In other embodiments, the particles have a skeletal density of about 1.15 to about 1.60 g/cm³. In still other embodiments, the particles have a skeletal density of about 1.25 to about 1.50 g/cm³. In certain other embodiments, the particles have a skeletal density of about 1.30 to about 1.40 g/cm³.

In other embodiments, the particles have a glass transition temperature that is higher than about 60° C. In some embodiments, the particles have a glass transition temperature that is higher than about 90° C. In still other embodiments, the particles have a glass transition temperature that is higher than about 100° C. In certain other embodiments, the particles have a glass transition temperature that is higher than about 130° C. In certain embodiments, the particles have a glass transition temperature that is higher than about 170° C.

In certain embodiments, the particles further comprise a carbohydrate, a protein stabilizer, an emulsifier, an amino acid, a surfactant, or a combination thereof.

In some embodiments, the carbohydrate is dextran, trehalose, sucrose, agarose, mannitol, lactose, sorbitol, maltose, or a combination thereof.

In other embodiments, the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethyl starch, N-methyl-2-pyrrolidone, sorbitol, sucrose, mannitol, cyclodextrin, hydroxypropyl beta-cyclodextrin, sulfobutylether beta-cyclodextrin, or a combination thereof. In preferred embodiments, the PEG is PEG 200, PEG 300, PEG 3350, PEG 8000, PEG 10000, PEG 20000, or a combination thereof.

In some embodiments, the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

In other embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

In some embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof.

In other embodiments, the particles have a surfactant content of less than about 10% by mass. In some embodiments, the particles have a surfactant content of less than about 5% by mass. In still other embodiments, the particles have a surfactant content of less than about 3% by mass. In certain other embodiments, the particles have a surfactant content of less than about 0.1% by mass. In certain preferred embodiments, the particles are substantially free from any surfactant content. In preferred embodiments, the particles have a surfactant content of less than about 1% by mass.

In some embodiments, the particles have less than about 7% residual moisture by weight. In other embodiments, the particles have less than about 5% residual moisture by weight. In certain embodiments, the particles have less than about 3% residual moisture by weight. In preferred embodiments, the particles have less than about 1% residual moisture by weight.

In other embodiments, the liquid is an organic solvent or an ionic liquid. In some embodiments, the organic solvent is benzyl benzoate, coconut oil, cottonseed oil, fish oil, grape seed oil, hazelnut oil, hydrogenated vegetable oils, olive oil, palm seed oil, peanut oil, peppermint oil, safflower oil, sesame oil, soybean oil, sunflower oil, walnut oil, acetone, ethyl acetate, ethyl lactate, dimethylacetamide, dimethyl isosorbide, dimethyl sulfoxide, glycofurol, diglyme, methyl tert-butyl ether, N-methyl pyrrolidone, perfluorodecalin, polyethylene glycol, 2-pyrrolidone, tetrahydrofurfuryl alcohol, trigylcerides, triglycerides of the fractionated plant fatty acids C8 and C10, propylene glycol diesters of saturated plant fatty acids C8 and C10, ethyl oleate, ethyl caprate, dibutyl adipate, fatty acid esters, hexanoic acid, octanoic acid, triacetin, diethyl glycol monoether, gamma-butyrolactone, eugenol, clove bud oil, citral, limonene, polyoxyl 40 hydrogenated castor oil, polyoxyl 35 castor oil, simple alcohols such as ethanol, octanol, hexanol, decanol, propanol, and butanol, gamma-butyrolactone, tocopherol, octa-fluoropropane, (perfluorohexyl)octane, n-acetyltryptophan, ethyl laurate, methyl caprylate, methyl caprate, methyl myristate, methyl oleate, methyl linoleate, dimethyl adipate, dibutyl suberate, diethyl sebacate, ethyl macadamiate, trimethylolpropane triisosterate, isopropyl laurate, isopropyl myristate, diethyl succinate, polysorbate esters, ethanol amine, propanoic acid, citral, anisole, anethol, benzaldehyde, linalool, caprolactone, phenol, thioglycerol, dimethylacetamide, diethylene glycol monoethyl ether, propylene carbonate, solketal, isosorbide dimethyl ether, ethyl formate, and ethyl hexyl acetate, or a combination thereof. In preferred embodiments, the organic solvent is ethyl oleate, trigylcerides, e.g., Miglyol, ethyl laureate, ethyl macadamiate, ethyl caprate, diethyl succinate, diethylene glycol monoethyl ether, propylene carbonate, or a combination thereof. In certain preferred embodiments, the organic solvent is ethyl oleate or trigylcerides.

Exemplary ionic liquids of the disclosure contain (i) cations such as pyridinium, pyridazinium, pyrimidinium, pyrazinium, imidazolium, pyrazolium, thiazolium, oxazolium, triazolium, ammonium, sulfonium; and (ii) anions such as halides, sulfates, sulfonates, carbonates, phosphates, bicarbonates, nitrates, acetates, PF₆—, BF₄—, triflate, nonaflate, bis(triflyl)amide, trifluoroacetate, heptafluorobutanoate, haloaluminate, or a combination thereof. In certain embodiments, the ionic liquid comprises pyridinium, pyridazinium, pyrimidinium, pyrazinium, imidazolium, pyrazolium, thiazolium, oxazolium, triazolium, ammonium, sulfonium, halides, sulfates, sulfonates, carbonates, phosphates, bicarbonates, nitrates, acetates, PF₆—, BF₄—, triflate, nonaflate, bis(trifyl)amide, trifluoroacetate, heptafluorobutanoate, haloaluminate, or a combination thereof.

In certain embodiments, the organic solvent is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof.

In some embodiments, the liquid further comprises a carbohydrate, a pH adjusting agent, a salt, a chelator, a mineral, a polymer, a surfactant, a protein stabilizer, an emulsifier, an antiseptic, an amino acid, an antioxidant, a protein, an organic solvent, a paraben, a bactericide, a fungicide, a vitamin, a preservative, a nutrient media, analgesic, or a combination thereof. In preferred embodiments, the liquid further comprises a carbohydrate, a pH adjusting agent, a salt, a surfactant, a protein stabilizer, an emulsifier, an amino acid, or a combination thereof. In certain preferred embodiments, the liquid further comprises a carbohydrate, a pH adjusting agent, a salt, a surfactant, a protein stabilizer, an emulsifier, an amino acid, or a combination thereof.

In other embodiments, the carbohydrate is dextran, trehalose, sucrose, agarose, mannitol, lactose, sorbitol, maltose, or a combination thereof.

In some embodiments, the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof.

In other embodiments, the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, magnesium chloride, potassium nitrate, or a combination thereof.

In some embodiments, the chelator is disodium edetate, ethylenediaminetetraacetic acid or pentetic acid. In other embodiments, the mineral is calcium, zinc, titanium dioxide, or a combination thereof. In certain embodiments, the polymer is propyleneglycol, glucose star polymer, silicone polymer, polydimethylsiloxane, polyethylene glycol, carboxymethylcellulose, poly(glycolic acid), poly(lactic-co-glycolic acid), polylactic acid, polycaprolactone (PCL), polyvinylpyrrolidone (PVP), ficoll, dextran, or a combination thereof.

In other embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, sodium laureth sulfate, lecithin, or a combination thereof. In certain embodiments, the surfactant is polysorbate. In preferred embodiments, the surfactant is polysorbate 20 or polysorbate 80.

In some embodiments, the protein stabilizer is acetyltryptophanate, caprylate, N-acetyltryptophan, trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, or a combination thereof. In certain embodiments, the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethyl starch, N-methyl-2-pyrrolidone, sorbitol, sucrose, mannitol, cyclodextrin, saccharides, or a combination thereof. In preferred embodiments, the protein stabilizer is trehalose, polyethylene glycol (PEG), cyclodextrin, hydroxypropyl beta-cyclodextrin, sulfobutylether beta-cyclodextrin, or a combination thereof. In certain preferred embodiments, the PEG is PEG 200, PEG 300, PEG 3350, PEG 8000, PEG 10000, PEG 20000, or a combination thereof. The stabilizers, used synonymously with the term “stabilizing agent”, as described herein, can be a salt, a carbohydrate, saccharides or amino acids, preferably a carbohydrate or saccharide admitted by the authorities as a suitable additive or excipient in pharmaceutical compositions. The term “stabilizer” refers to an excipient or a mixture of excipients which stabilizes the physical and/or chemical properties of biologically active peptides. In some embodiments, stabilizers prevent, e.g., degradation of the biologically active peptides during droplet formation, desiccation, and/or storage of the particulate matter. Exemplary stabilizers include, but are not limited to, sugars, salts, hydrophobic salts, detergents, reducing agents, cyclodextrins, polyols, carboxylic acids, and amino acids. A “stable” formulation as described herein, refers to a formulation in which the biologically active peptide retains an acceptable portion of its essential physical, chemical, or biological properties over an acceptable period of time. In the case of proteins and peptides, e.g., exemplary methods of assessing stability are reviewed in (i) Peptide and Protein Drug Delivery, 247-301, Vincent Lee Ed., Marcel Dekker, Inc., New York, NY, 1991, and (ii) Jones, A., Adv. Drug Delivery Rev. 10: 29-90 (1993). In certain embodiments, chemical stability of a protein is assessed by measuring the size distribution of the sample at several stages. These include, e.g., before particle formation (assessment of the feed solution), immediately after particle formation, and again after a period of storage, where storage takes place either within or in the absence of a suspension formulation carrier medium. In certain other embodiments, the size distribution is assessed by size exclusion chromatography (SEC-HPLC).

Examples of emulsifiers suitable for use in the liquid include, but are not limited to, lipophilic agents having an HLB of less than 7, such as mixed fatty acid monoglycerides; mixed fatty acid diglycerides; mixtures of fatty acid mono- and diglycerides; lipophilic polyglycerol esters; glycerol esters including glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; and mixtures thereof glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; or a combination thereof. In some embodiments, the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof. In preferred embodiments, the emulsifier is polysorbate 80, sorbitan monooleate, or a combination thereof.

In other embodiments, the antiseptic is phenol, m-cresol, benzyl alcohol, 2-phenyloxyethanol, chlorobutanol, neomycin, benzethonium chloride, gluteraldehyde, beta-propiolactone, or a combination thereof.

In certain embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof. In preferred embodiments, the amino acid is arginine, histidine, proline, asparagine, or a combination thereof.

In some embodiments, the antioxidant is glutathione, ascorbic acid, cysteine, N-acetyl-L-tryptophanate, tocopherol, histidine, methionine, tocopherol, or a combination thereof. In other embodiments, the protein is protamine, protamine sulfate, gelatin, or a combination thereof. In certain embodiments, the organic solvent is dimethyl sulfoxide, N-methyl-2-pyrrolidone, or a combination thereof. In still other embodiments, the preservative is methyl hydroxybenzoate, thimerosal, a paraben, formaldehyde, castor oil, or a combination thereof. In certain other embodiments, the preservative is sodium nitrate, sulfur dioxide, potassium sorbate, sodium sorbate, sodium benzoate, benzoic acid, methyl hydroxybenzoate, thimerosal, parabens, formaldehyde, castor oil, or a combination thereof. The paraben can be a parahydroxybenzoate. In some embodiments, the bactericide is benzalkonium chloride (cationic surfactants), hypochlorites, peroxides, alcohols, phenolic compounds (e.g. carbolic acid), or a combination thereof.

In other embodiments, the fungicide is acibenzolar, 2-phenylphenol, anilazine, carvone, natamycin, potassium azide, or a combination thereof. In certain embodiments, the vitamin is thiamine, riboflavin, niacin, pantothenic acid, biotin, vitamin B6, vitamin B12, folate, niacin, ascorbic acid, calciferols, retinols, quinones, or a combination thereof.

A number of nutrient media, preferably serum free, alone or in combination, may be used in the present disclosure, including commercially available media or other media well known in the art. Examples of such media (all without serum or having had the serum removed) include ADC-1, LPM (Bovine Serum Albumin-free), F10 (HAM), F12 (HAM), DCCM1, DCCM2, RPMI 1640, BGJ Medium (Fitton-Jackson Modification), Basal Medium Eagle (BME-with the addition of Earle's salt base), Dulbecco's Modified Eagle Medium (DMEM-without serum), Glasgow Modification Eagle Medium (GMEM), Leibovitz L-15 Medium, McCoy's 5 A Medium, Medium M199 (M199E-with Earle's salt base), Medium M199 (M199H- with Hank's salt base), Minimum Essential Medium Eagle (MEM-E- with Earle's salt base), Minimum Essential Medium Eagle (MEM-H- with Hank's salt base) and Minimum Essential Medium Eagle (MEM-NAA- with non-essential amino acids), among numerous others. In addition, serum-containing nutrient media may also be used in compositions according to the present disclosure, but the use of serum-containing media is less preferred because of the possibility that the serum may be contaminated with microbial agents and because the patient may develop immunological reactions to certain antigenic components contained in the serum.

In some embodiments, the analgesic is paracetamol, histamine receptor antagonist (e.g., an H1 or an H2 blocker), NSAIDs, COX-2 inhibitors, Celecoxib, Rofecoxib, Valdecoxib, Parecoxib, Lumiracoxib, Etoricoxib, Firocoxib, acetaminophen, opiates, Dextropropoxyphene, Codeine, Tramadol, Anileridine, Pethidine, Hydrocodone, Morphine, Oxycodone, Methadone, Diacetylmorphine, Hydromorphone, Oxymorphone, Levorphanol, Buprenorphine, Fentanyl, Sufentanyl, Etorphine, Carfentanil, dihydromorphine, dihydrocodeine, Thebaine, Papaverine, diproqualone, Flupirtine, Tricyclic antidepressants, Acetaminophen or lidocaine, or a combination thereof. In certain embodiments, the analgesic is acetaminophen or lidocaine.

In certain embodiments, the liquid further comprises at least one pharmaceutically acceptable additive, diluent, excipient, carrier, or a combination thereof.

In other embodiments, the composition has a viscosity of less than about 200 mPa·s, less than about 150 mPa·s, less than about 125 mPa·s, less than about 100 mPa·s, less than about 75 mPa·s, less than about 75 mPa·s, less than about 70 mPa·s, less than about 65 mPa·s, less than about 60 mPa·s, less than about 55 mPa·s, less than about 50 mPa·s, less than about 45 mPa·s, less than about 40 mPa·s, less than about 35 mPa·s, less than about 30 mPa·s, less than about 25 mPa·s, less than about 20 mPa·s, less than about 19 mPa·s, less than about 18 mPa·s, less than about 17 mPa·s, less than about 16 mPa·s, less than about 15 mPa·s, less than about 14 mPa·s, less than about 13 mPa·s, less than about 12 mPa·s, less than about 11 mPa·s, less than about 10 mPa·s, less than about 9.5 mPa·s, less than about 9 mPa·s, less than about 8.5 mPa·s, less than about 8 mPa·s, less than about 7.5 mPa·s, less than about 7 mPa·s, less than about 6.5 mPa·s, less than about 6 mPa·s, less than about 5.5 mPa·s, less than about 5 mPa·s, less than about 4.5 mPa·s, less than about 4 mPa·s, less than about 3.5 mPa·s, less than about 3 mPa·s, less than about 2.5 mPa·s, less than about 2 mPa·s, less than about 1.5 mPa·s, less than about 1 mPa·s, less than about 0.5 mPa·s, less than about 0.1 mPa·s, less than about 0.05 mPa·s, or less than about 0.01 mPa·s (one millipascal-second). In other embodiments, the composition has a viscosity of about 0.01 mPa·s to about 10,000 mPa·s, e.g., from about 0.01 mPa·s to about 1,000 mPa·s, from about 0.01 mPa·s to about 100 mPa·s, from about 0.01 mPa·s to about 50 mPa·s, from about 0.01 mPa·s to about 25 mPa·s, from about 0.01 mPa·s to about 10 mPa·s, from about 0.01 mPa·s to about 5 mPa·s, or from about 0.01 mPa·s to about 1 mPa·s. In certain embodiments, the viscosity of the composition can range from about 0.27 mPa·s to about 200 mPa·s, e.g., about 0.27 mPa·s to about 50 mPa·s, about 1 mPa·s to about 30 mPa·s, or about 20 mPa·s to about 50 mPa·s. In still other embodiments, the viscosity of the composition ranges from about 0.27 mPa·s to about 200 mPa·s, e.g., about 0.27 mPa·s to about 100 mPa·s, about 0.27 mPa·s to about 50 mPa·s, about 0.27 mPa·s to about 30 mPa·s, about 1 mPa·s to about 20 mPa·s, or about 1 mPa·s to about 15 mPa·s. The term “viscosity” is used to describe the property of a fluid acting to resist shearing flow. For the purposes of the present disclosure, viscosity can be determined using a rheometer, e.g., AR-G2 Rheometer (TA Instruments, USA), fitted with a cone and plate (2°/40 mm) at 25° C. at a specified shear rate. In certain embodiments, the viscosity is measured at a shear rate in the Newtonian regime. The term “Newtonian regime” means a range of shear rates which are linearly proportional or nearly linearly proportional to the local strain rate at every point. In some embodiments, the viscosity is measured at a shear rate of about 100 s⁻¹ or greater, e.g., at about 1000 s⁻¹ or greater than about 1000 s⁻¹. The composition may include from about 5 to about 90% particles by volume, e.g., e.g., about 20 to about 90%, about 40 to about 80%, about 50 to about 60%, or about 70 to about 90%. The composition may have a concentration of the peptide from about 0.0001 to about 1000 mg/mL, e.g., from about 100 to about 900, about 150 to about 800, or about 200 to about 700 mg/mL. Methods of controlling viscosity include temperature regulation and viscosity modifying additives. Mixtures of liquids may also be used to control viscosity. The units “mPa·s” and “cP” are used herein, interchangeably in the broadest sense.

In some embodiments, the composition has a viscosity of less than about 200 mPa·s. In other embodiments, the composition has a viscosity of less than about 150 mPa·s. In still other embodiments, the composition has a viscosity of less than about 50 mPa·s. In other embodiments, the composition has a viscosity of less than about 30 mPa·s. In certain other embodiments, the composition has a viscosity of less than about 20 mPa·s. In certain embodiments, the composition has a viscosity of less than about 10 mPa·s. In other embodiments, the composition has a viscosity of less than about 5 mPa·s. In certain preferred embodiments, the composition has a viscosity of less than about 3 mPa·s. In preferred embodiments, the composition has a viscosity of less than about 2.5 mPa·s.

In preferred embodiments according to the disclosure as described herein, the composition comprising a plurality of particles has improved stability of the biologically active peptide compared to an aqueous composition comprising the biologically active peptide in monomeric form.

The particles comprising at least one peptide described herein, can be used in a number of ways, as well as any methods for the delivery of the particles disclosed in, for example, U.S. Appl. No. 62/899,907 and U.S. Appl. No. 62/899,981, each of which is hereby incorporated by reference in its entirety.

### Methods of the Disclosure

The methods described herein, are generally provided for forming particles, the method comprising: a) providing droplets comprising a first liquid and a peptide; b) contacting the droplets comprising the peptide with a second liquid; c) allowing the droplets to dry; and d) removing the first and second liquids, thereby forming particles comprising a peptide, wherein the particles comprise up to about 10% (w/w) of peptide and the circularity of the particles is from about 0.10 to about 1.00 after removing the first and second liquids. In certain embodiments, the peptide is a biologically active peptide. In preferred embodiments, the biologically active peptide comprises from about 2 to about 12 amino acid residues.

In other embodiments, the particles comprise up to about 8% (w/w) of peptide. In still other embodiments, the particles comprise up to about 6% (w/w) of peptide. In certain other embodiments, the particles comprise up to about 5% (w/w) of peptide. In certain embodiments, the particles comprise up to about 3% (w/w) of peptide.

In some embodiments, the circularity of the particles is from about 0.80 to about 1.00 after removing the first and second liquids. In other embodiments, the circularity of the particles is from about 0.85 to about 1.00 after removing the first and second liquids. In still other embodiments, the circularity of the particles is from about 0.90 to about 1.00 after removing the first and second liquids. In certain other embodiments, the circularity of the particles is from about 0.95 to about 1.00 after removing the first and second liquids. In certain preferred embodiments, the circularity of the particles is from about 0.98 to about 1.00 after removing the first and second liquids. In preferred embodiments, the circularity of the particles is about 1.00 after removing the first and second liquids.

In preferred embodiments, the particles have a substantially smooth surface after removing the first and second liquids.

In other embodiments, the particles have a diameter of about 0.1 to about 100 μm after removing the first and second liquids. In some embodiments, the particles have a diameter of about 1 to about 100 μm after removing the first and second liquids. In still other embodiments, the particles have a diameter of about 5 to about 100 μm after removing the first and second liquids. In certain other embodiments, the particles have a diameter of about 5 to about 50 μm after removing the first and second liquids. In certain embodiments, the particles have a diameter of about 5 to about 20 μm after removing the first and second liquids.

In some embodiments, the particles have a skeletal density of about 1.00 to about 6.00 g/cm³ after removing the first and second liquids. In other embodiments, the particles have a skeletal density of about 1.15 to about 1.60 g/cm³ after removing the first and second liquids. In still other embodiments, the particles have a skeletal density of about 1.25 to about 1.50 g/cm³ after removing the first and second liquids. In certain other embodiments, the particles have a skeletal density of about 1.30 to about 1.40 g/cm³ after removing the first and second liquids.

In other embodiments, the particles have a glass transition temperature that is higher than about 60° C. after removing the first and second liquids. In some embodiments, the particles have a glass transition temperature that is higher than about 90° C. after removing the first and second liquids. In still other embodiments, the particles have a glass transition temperature that is higher than about 100° C. after removing the first and second liquids. In certain other embodiments, the particles have a glass transition temperature that is higher than about 130° C. after removing the first and second liquids. In certain embodiments, the particles have a glass transition temperature that is higher than about 170° C. after removing the first and second liquids.

In certain embodiments, the particles further comprise a carbohydrate, a protein stabilizer, an emulsifier, an amino acid, a surfactant, or a combination thereof.

In some embodiments, the carbohydrate is dextran, trehalose, sucrose, agarose, mannitol, lactose, sorbitol, maltose, or a combination thereof.

In other embodiments, the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethyl starch, N-methyl pyrrolidone, sorbitol, sucrose, mannitol, cyclodextrin, hydroxypropyl beta-cyclodextrin, sulfobutylether beta-cyclodextrin, or a combination thereof. In preferred embodiments, the PEG is PEG 200, PEG 300, PEG 3350, PEG 8000, PEG 10000, PEG 20000, or a combination thereof.

In some embodiments, the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

In other embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

In some embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof.

In other embodiments, the particles have a surfactant content of less than about 10% by mass remaining after removing the first and second liquids. In some embodiments, the particles have a surfactant content of less than about 5% by mass remaining after removing the first and second liquids. In still other embodiments, the particles have a surfactant content of less than about 3% by mass remaining after removing the first and second liquids. In certain other embodiments, the particles have a surfactant content of less than about 0.1% by mass remaining after removing the first and second liquids. In certain preferred embodiments, the particles are substantially free from any surfactant content after removing the first and second liquids. In preferred embodiments, the particles have a surfactant content of less than about 1% by mass remaining after removing the first and second liquids.

In some embodiments, the particles have less than about 7% of residual first and second liquid by mass remaining after removing the first and second liquids. In other embodiments, the particles have less than about 5% of residual first and second liquid by mass remaining after removing the first and second liquids. In certain embodiments, the particles have less than about 3% of residual first and second liquid by mass remaining after removing the first and second liquids. In preferred embodiments, the particles have less than about 1% of residual first and second liquid by mass remaining after removing the first and second liquids.

In other embodiments, the particle has less than about 7% residual moisture by weight after removing the first and second liquids. In still other embodiments, the particle has less than about 5% residual moisture by weight after removing the first and second liquids. In certain embodiments, the particle has less than about 3% residual moisture by weight after removing the first and second liquids. In preferred embodiments, the particle has less than about 1% residual moisture by weight after removing the first and second liquids.

Droplets as described herein, can be formed through any of several techniques that are known in the art. These include rotary atomization, pneumatic atomization, ultrasonic atomization, sonic atomization, vibrating mesh nebulization, jet atomization, microfluidic droplet generation, flow focusing, membrane emulsification, electrospray, or homogenization. The term “droplet” or “droplets” or “drops” refer to a material that has a liquid outer surface. In certain embodiments, the droplets of step a) are formed by electrospray, an ultrasonic atomizer, or a microfluidic device. In preferred embodiments, the droplets of step a) are formed by an ultrasonic atomizer or by vortex.

The term “feed solution” refers to a preparation of the peptides in the first liquid, either as a solution, a slurry, or some other liquid form. In some embodiments, the preparation contains excipients. In other embodiments, the preparation further contains a buffer.

In some embodiments, the first liquid is an aqueous liquid, an organic solvent, an ionic liquid, a hydrogel, an ionogel, or a combination thereof. In other embodiments, the first liquid is an aqueous liquid. In certain embodiments, the first liquid is water, 0.9% saline, lactated Ringer's solution, a buffer, dextrose 5%, or a combination thereof. In certain other embodiments, the buffer is acetate buffer, histidine buffer, succinate buffer, HEPES buffer, tris buffer, carbonate buffer, citrate buffer, phosphate buffer, phosphate-buffered saline, glycine buffer, barbital buffer, cacodylate buffer, ammonium formate buffer, urea solution, or a combination thereof. In preferred embodiments, the first liquid is water.

In other embodiments, the organic liquid is acetone, acetonitrile, acyclic alkanes (e.g., hexanes, heptane, pentane), amyl acetate, butanol, butyl acetate, chlorobenzene, chloroform, cumene, cyclohexane, 1,2-dichloroethene, dichloromethane, diethyl ether, dimethoxyethane, dimethylacetamide, dimethylformamide, dimethyl sulfoxide, 1,4-dioxane, ethanol, 2-ethoxyethanol, ethyl acetate, ethyl nitrate, ethyleneglycol, hydrazine, isopropanol, methanol, methyl acetate, 2-methyl-1-butanol, 2-methyl-1-propanol, methylbutyl ketone, methylcyclohexane, methylethyl ketone, methylpyrrolidone, methyl tert-butyl ether, nitromethane, propanol, propyl acetate, sulfolane, propyleneglycol, tetrahydrofuran, tetralin, toluene, 1,1,2-tricholoroethane, triethylamine, xylene, benzyl benzoate, ethyl lactate, dimethyl isosorbide, dimethyl sulfoxide, glycofurol, diglyme, methyl tert-butyl ether, polyethylene glycol, 2-pyrrolidone, tetrahydrofurfuryl alcohol, trigylcerides, octyl acetate, ethanol, butanol, octanol, decanol, diglyme, tocopherol, octa-fluoropropane, (perfluorohexyl)octane, n-acetyltryptophan, trigylcerides, triglycerides of the fractionated plant fatty acids C8 and C10, propylene glycol diesters of saturated plant fatty acids C8 and C10, ethyl laurate, methyl caprylate, methyl caprate, methyl myristate, methyl oleate, methyl linoleate, dimethyl adipate, dibutyl suberate, diethyl sebacate, ethyl macadamiate, trimethylolpropane triisosterate, isopropyl laurate, isopropyl myristate, diethyl succinate, polysorbate esters, ethanol amine, propanoic acid, triacetin, citral, anisole, anethol, benzaldehyde, linalool, caprolactone, phenol, thioglycerol, dimethylacetamide, ethyl formate, ethyl hexyl acetate, eugenol, clove bud oil, diethyl glycol monoether, dimethyl isosorbide, isopropyl acetate, methyl isobutyl ketone, methyl tert-butyl ether, N-methyl pyrrolidone, perfluorodecalin, 2-pyrrolidone, ethyl oleate, ethyl caprate, dibutyl adipate, hexanoic acid, octanoic acid, diethyl glycol monoether, gamma-butyrolactone, polyoxyl 40 hydrogenated castor oil, polyoxyl 35 castor oil, propylene carbonate, octanol, hexanol, sorbitan monooleate, n-acetyltryptophan, solketal, an alkyl acetate, an aryl acetate, an aryl alkyl acetate, tolyl acetate, benzyl acetate, polysorbate 80, phenethyl acetate, phenyl acetate, glycerol, or a combination thereof. In other embodiments, the first liquid is an oil. In certain embodiments, the oil is coconut oil, cottonseed oil, fish oil, grape seed oil, hazelnut oil, hydrogenated vegetable oils, lime oil, olive oil, palm seed oil, peanut oil, peppermint oil, safflower oil, sesame oil, soybean oil, sunflower oil, walnut oil, silicon oil, mineral oil, or a combination thereof. In still other embodiments, the first liquid is an ionic liquid. In certain other embodiments, the ionic liquid contains (i) cations such as pyridinium, pyridazinium, pyrimidinium, pyrazinium, imidazolium, pyrazolium, thiazolium, oxazolium, triazolium, ammonium, sulfonium; and (ii) anions such as halides, sulfates, sulfonates, carbonates, phosphates, bicarbonates, nitrates, acetates, PF₆—, BF₄—, triflate, nonaflate, bis(triflyl)amide, trifluoroacetate, heptafluorobutanoate, haloaluminate, or a combination thereof.

In certain embodiments, the first liquid is a hydrogel, an ionogel, or a combination thereof. Exemplary hydrogels are prepared from polymers such as collagen, chitosan, methylcellulose, dextran, alginate, agarose, poly(methyl methacrylate), poly(amido amine), poly(ethyleneimine), polyethylene oxide, gelatin, hyaluronic acid, or a combination thereof, and may contain water, aqueous solutions, and other polar solvents. Exemplary organogels are prepared form organogelators such as 4-tert-butyl-1-aryl cyclohexanols, L-lysine derivatives, poly(ethylene glycol), polycarbonate, polyesters, polyalkenes, oxalyl amide derivatives containing alkyl ester groups, or low molecular weight compounds such as fatty acids and n-alkanes, and contain a non-polar solvent phase. Ionogels are analogous to organogels with the exception that the solvent phase is an ionic liquid.

In some embodiments, the concentration of the peptide in the first liquid as described herein, is about 10 mg/mL to about 650 mg/mL, e.g., about 20, 30, 50, 75, 100, 125, 150, 175, 200, 225, 250, 275, 300, 325, 350, 375, 400, 425, 450, 475, 500, 525, 550, 575, 600, 625 mg/mL to about 650 mg/mL; about 20 mg/mL to about 625 mg/mL, e.g., about 20, 30, 50, 75, 100, 125, 150, 175, 200, 225, 250, 275, 300, 325, 350, 375, 400, 425, 450, 475, 500, 525, 550, 575, 600 mg/mL to about 625 mg/mL; about 20 mg/mL to about 600 mg/mL, e.g., about 20, 30, 50, 75, 100, 125, 150, 175, 200, 225, 250, 275, 300, 325, 350, 375, 400, 425, 450, 475, 500, 525, 550, 575 mg/mL to about 600 mg/mL; about 20 mg/mL to about 575 mg/mL, e.g., about 20, 30, 50, 75, 100, 125, 150, 175, 200, 225, 250, 275, 300, 325, 350, 375, 400, 425, 450, 475, 500, 525, 550 mg/mL to about 575 mg/mL; about 20 mg/mL to about 550 mg/mL, e.g., about 20, 30, 50, 75, 100, 125, 150, 175, 200, 225, 250, 275, 300, 325, 350, 375, 400, 425, 450, 475, 500, 525 mg/mL to about 550 mg/mL; about 20 mg/mL to about 525 mg/mL, e.g., about 20, 30, 50, 75, 100, 125, 150, 175, 200, 225, 250, 275, 300, 325, 350, 375, 400, 425, 450, 475, 500 mg/mL to about 525 mg/mL; about 20 mg/mL to about 500 mg/mL, e.g., about 20, 30, 50, 75, 100, 125, 150, 175, 200, 225, 250, 275, 300, 325, 350, 375, 400, 425, 450, 475 mg/mL to about 500 mg/mL; about 20 mg/mL to about 475 mg/mL, e.g., about 20, 30, 50, 75, 100, 125, 150, 175, 200, 225, 250, 275, 300, 325, 350, 375, 400, 425, 450 mg/mL to about 475 mg/mL; about 20 mg/mL to about 450 mg/mL, e.g., about 20, 30, 50, 75, 100, 125, 150, 175, 200, 225, 250, 275, 300, 325, 350, 375, 400, 425 mg/mL to about 450 mg/mL; about 20 mg/mL to about 425 mg/mL, e.g., about 20, 30, 50, 75, 100, 125, 150, 175, 200, 225, 250, 275, 300, 325, 350, 375, 400 mg/mL to about 425 mg/mL; about 20 mg/mL to about 400 mg/mL, e.g., about 20, 30, 50, 75, 100, 125, 150, 175, 200, 225, 250, 275, 300, 325, 350, 375 mg/mL to about 400 mg/mL; about 20 mg/mL to about 375 mg/mL, e.g., about 20, 30, 50, 75, 100, 125, 150, 175, 200, 225, 250, 275, 300, 325, 350 mg/mL to about 375 mg/mL; about 20 mg/mL to about 350 mg/mL, e.g., about 20, 30, 50, 75, 100, 125, 150, 175, 200, 225, 250, 275, 300, 325 mg/mL to about 350 mg/mL; about 20 mg/mL to about 325 mg/mL, e.g., about 20, 30, 50, 75, 100, 125, 150, 175, 200, 225, 250, 275, 300 mg/mL to about 325 mg/mL; or about 20 mg/mL to about 300 mg/mL, e.g., about 20, 30, 50, 75, 100, 125, 150, 175, 200, 225, 250, 275 mg/mL to about 300 mg/mL. In other embodiments, the concentration of the peptide in the first liquid is about 10 mg/mL to about 500 mg/mL. In certain embodiments, the concentration of the peptide in the first liquid is about 10 mg/mL to about 100 mg/mL. In preferred embodiments, the concentration of the peptide in the first liquid is about 3 mg/mL to about 25 mg/mL. In other embodiments of the disclosure, the concentration of the peptide in the first liquid is from about 0.0001 mg/mL to about 1000 mg/mL, e.g., about 100 to about 800, about 200 to about 700, about 200 to about 600, or about 300 to about 700 mg/mL. In still other embodiments, the particles have a mass loading of the peptide from about 1% to about 100%.

In certain embodiments, the first liquid further comprises a carbohydrate, a protein stabilizer, an emulsifier, an amino acid, a surfactant, or a combination thereof.

In some embodiments, the carbohydrate is dextran, trehalose, sucrose, agarose, mannitol, lactose, sorbitol, maltose, or a combination thereof.

In other embodiments, the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethyl starch, N-methyl-2-pyrrolidone, sorbitol, sucrose, mannitol, cyclodextrin, hydroxypropyl beta-cyclodextrin, sulfobutylether beta-cyclodextrin, or a combination thereof. In preferred embodiments, the PEG is PEG 200, PEG 300, PEG 3350, PEG 8000, PEG 10000, PEG 20000, or a combination thereof.

In some embodiments, the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

In other embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

In some embodiments, the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof.

In other embodiments, the first liquid has a viscosity of less than about 200 mPa·s, less than about 150 mPa·s, less than about 125 mPa·s, less than about 100 mPa·s, less than about 75 mPa·s, less than about 75 mPa·s, less than about 70 mPa·s, less than about 65 mPa·s, less than about 60 mPa·s, less than about 55 mPa·s, less than about 50 mPa·s, less than about 45 mPa·s, less than about 40 mPa·s, less than about 35 mPa·s, less than about 30 mPa·s, less than about 25 mPa·s, less than about 20 mPa·s, less than about 19 mPa·s, less than about 18 mPa·s, less than about 17 mPa·s, less than about 16 mPa·s, less than about 15 mPa·s, less than about 14 mPa·s, less than about 13 mPa·s, less than about 12 mPa·s, less than about 11 mPa·s, less than about 10 mPa·s, less than about 9.5 mPa·s, less than about 9 mPa·s, less than about 8.5 mPa·s, less than about 8 mPa·s, less than about 7.5 mPa·s, less than about 7 mPa·s, less than about 6.5 mPa·s, less than about 6 mPa·s, less than about 5.5 mPa·s, less than about 5 mPa·s, less than about 4.5 mPa·s, less than about 4 mPa·s, less than about 3.5 mPa·s, less than about 3 mPa·s, less than about 2.5 mPa·s, less than about 2 mPa·s, less than about 1.5 mPa·s, less than about 1 mPa·s, less than about 0.5 mPa·s, less than about 0.1 mPa·s, less than about 0.05 mPa·s, or less than about 0.01 mPa·s (one millipascal-second). In other embodiments, the first liquid has a viscosity of about 0.01 mPa·s to about 10,000 mPa·s, e.g., from about 0.01 mPa·s to about 1,000 mPa·s, from about 0.01 mPa·s to about 100 mPa·s, from about 0.01 mPa·s to about 50 mPa·s, from about 0.01 mPa·s to about 25 mPa·s, from about 0.01 mPa·s to about 10 mPa·s, from about 0.01 mPa·s to about 5 mPa·s, or from about 0.01 mPa·s to about 1 mPa·s. In certain embodiments, the first liquid has a viscosity that can range from about 0.27 mPa·s to about 200 mPa·s, e.g., about 0.27 mPa·s to about 50 mPa·s, about 1 mPa·s to about 30 mPa·s, or about 20 mPa·s to about 50 mPa·s. In still other embodiments, the first liquid has a viscosity that ranges from about 0.27 mPa·s to about 200 mPa·s, e.g., about 0.27 mPa·s to about 100 mPa·s, about 0.27 mPa·s to about 50 mPa·s, about 0.27 mPa·s to about 30 mPa·s, about 1 mPa·s to about 20 mPa·s, or about 1 mPa·s to about 15 mPa·s. Methods of controlling viscosity include temperature regulation and viscosity modifying additives. Mixtures of liquids may also be used to control viscosity.

In some embodiments, the first liquid has a viscosity from about 0.01 to about 10,000 mPa·s. In other embodiments, the first liquid has a viscosity of less than about 100 mPa·s. In still other embodiments, the first liquid has a viscosity of less than about 10 mPa·s. In certain other embodiments, the first liquid has a viscosity of less than about 3 mPa·s. In certain embodiments, the first liquid has a viscosity of less than about 0.9 mPa·s.

In other embodiments, the second liquid is an aqueous liquid, an organic solvent, an ionic liquid, a hydrogel, ionogel, protein stabilizer, or a combination thereof. In preferred embodiments, the second liquid is an organic solvent.

In some embodiments, the organic solvent is benzyl alcohol, benzyl benzoate, castor oil, coconut oil, corn oil, cottonseed oil, fish oil, grape seed oil, hazelnut oil, hydrogenated palm seed oil, olive oil, peanut oil, peppermint oil, safflower oil, sesame oil, soybean oil, sunflower oil, vegetable oil, walnut oil, polyethylene glycol, glycofurol, acetone, diglyme, dimethylacetamide, dimethyl isosorbide, dimethyl sulfoxide, ethanol, ethyl acetate, butyl acetate, ethyl ether, ethyl lactate, isopropyl acetate, methyl acetate, methyl isobutyl ketone, methyl tert-butyl ether, N-methyl pyrrolidone, perfluorodecalin, 2-pyrrolidone, trigylcerides, tetrahydrofurfuryl alcohol, triglycerides of the fractionated plant fatty acids C8 and C10 (e.g., MIGLYOL® 810 and MIGLOYL® 812N), propylene glycol diesters of saturated plant fatty acids C8 and C10 (e.g., MIGLYOL® 840), ethyl oleate, ethyl caprate, dibutyl adipate, fatty acid esters, hexanoic acid, octanoic acid, triacetin, diethyl glycol monoether, gamma-butyrolactone, eugenol, clove bud oil, citral, limonene, or a combination thereof. In certain embodiments, the organic solvent is ethyl acetate or butyl acetate.

In other embodiments, the organic solvent is acetone, acetonitrile, acyclic alkanes (e.g., hexanes, heptane, pentane), amyl acetate, butanol, butyl acetate, chlorobenzene, chloroform, cumene, cyclohexane, 1,2-dichloroethene, dichloromethane, diethyl ether, dimethoxyethane, dimethylacetamide, dimethylformamide, dimethyl sulfoxide, 1,4-dioxane, ethanol, 2-ethoxyethanol, ethyl acetate, ethyl nitrate, ethyleneglycol, hydrazine, isopropanol, methanol, methyl acetate, 2-methyl-1-butanol, 2-methyl-1-propanol, methylbutyl ketone, methylcyclohexane, methylethyl ketone, methylpyrrolidone, methyl tert-butyl ether, nitromethane, propanol, propyl acetate, sulfolane, propyleneglycol, tetrahydrofuran, tetralin, toluene, 1,1,2-tricholoroethane, triethylamine, xylene, benzyl benzoate, ethyl lactate, dimethyl isosorbide, dimethyl sulfoxide, glycofurol, diglyme, methyl tert-butyl ether, polyethylene glycol, 2-pyrrolidone, tetrahydrofurfuryl alcohol, trigylcerides, octyl acetate, ethanol, butanol, octanol, decanol, diglyme, tocopherol, octa-fluoropropane, (perfluorohexyl)octane, n-acetyltryptophan, trigylcerides, triglycerides of the fractionated plant fatty acids C8 and C10, propylene glycol diesters of saturated plant fatty acids C8 and C10, ethyl laurate, methyl caprylate, methyl caprate, methyl myristate, methyl oleate, methyl linoleate, dimethyl adipate, dibutyl suberate, diethyl sebacate, ethyl macadamiate, trimethylolpropane triisosterate, isopropyl laurate, isopropyl myristate, diethyl succinate, polysorbate esters, ethanol amine, propanoic acid, triacetin, citral, anisole, anethol, benzaldehyde, linalool, caprolactone, phenol, thioglycerol, dimethylacetamide, ethyl formate, ethyl hexyl acetate, eugenol, clove bud oil, diethyl glycol monoether, dimethyl isosorbide, isopropyl acetate, methyl isobutyl ketone, methyl tert-butyl ether, N-methyl pyrrolidone, perfluorodecalin, 2-pyrrolidone, ethyl oleate, ethyl caprate, dibutyl adipate, hexanoic acid, octanoic acid, diethyl glycol monoether, gamma-butyrolactone, polyoxyl 40 hydrogenated castor oil, polyoxyl 35 castor oil, propylene carbonate, octanol, hexanol, sorbitan monooleate, n-acetyltryptophan, solketal, an alkyl acetate, an aryl acetate, an aryl alkyl acetate, tolyl acetate, benzyl acetate, polysorbate 80, phenethyl acetate, phenyl acetate, glycerol, or a combination thereof.

In certain embodiments, the organic solvent is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof.

In certain other embodiments, the second liquid is an ionic liquid. In still other embodiments, the second liquid is a protein stabilizer.

In some embodiments, the second liquid has a viscosity from about 0.01 to about 10,000 mPa·s. In other embodiments, the second liquid has a viscosity of less than about 10 mPa·s. In still other embodiments, the second liquid has a viscosity of less than about 5 mPa·s. In certain other embodiments, the second liquid has a viscosity of less than about 2 mPa·s. In certain embodiments, the second liquid has a viscosity of less than about 0.70 mPa·s. In preferred embodiments, the second liquid has a viscosity of less than about 0.40 mPa·s. Methods of controlling viscosity include temperature regulation and viscosity modifying additives. Mixtures of liquids may also be used to control viscosity.

In other embodiments, the droplets of step a) are formed by electrospray, an ultrasonic atomizer, vortex, or a microfluidic device. In certain embodiments, the droplets of step a) are formed by an ultrasonic atomizer or by vortex.

### Formation of Particles

The particles as described herein, can be formed by placing droplets that include a first liquid and a peptide in contact with a second liquid that facilitates removal of the first liquid. In some embodiments, the droplets are formed in a separate medium and placed into contact with the second liquid thereafter, e.g., by dripping or spraying them into or onto the second liquid. In other embodiments, the droplets are formed within the second liquid, such that they are immediately in contact. Particle formation begins to take place when at least a subset of the components of the droplets begin to undergo precipitation or phase separation as the first liquid is removed from the droplets. In preferred embodiments, the droplets are dried after contacting the droplets comprising the peptide with a second liquid.

In some embodiments, particles are formed after the first liquid disperses throughout the second liquid, e.g., through a diffusion process. In other embodiments the second liquid may have varying degrees of miscibility with the first liquid and represent a weakly or negligibly solubilizing medium in relation to the components of the particles or a subset of the components of the particles, e.g., the peptides. The peptides are typically less soluble in the second liquid relative to the first liquid in the timeframe of or under the conditions disclosed herein, e.g., at least about 5, 10, 100, or about 1000 times less soluble.

In other embodiments as described herein, step b) further comprises decreasing the temperature of the second liquid to a temperature within about 30° C. of the freezing point of the first liquid. In certain other embodiments, the boiling point of the second liquid at atmospheric pressure is from about 0 to about 200° C. In still other embodiments, the second liquid is a mixture of two or more liquids of different polarities. In certain preferred embodiments, the mixture comprises liquids wherein the mixture comprises liquids having differing solubility.

The term “primary desiccation” refers to a step by which a droplet comprising a first liquid is placed in contact with a second liquid and dried or desiccated by the second liquid, e.g., through dispersal of the first liquid in the second liquid, and/or through evaporation.

The term “secondary desiccation” refers to a post-processing step, e.g., after removal of the first and second liquids by which the residual moisture and/or solvent content of the particles is modified. Exemplary methods of secondary desiccation include vacuum drying, with or without the application of heat, lyophilization, fluidized bed drying, tray drying, belt drying, or slurry spray drying. Secondary desiccation may also be used to remove any washing liquids that are used to separate the particles from the second liquid. In preferred embodiments, the first and second liquids are removed through centrifugation, sieving, filtration, magnetic collection, solvent exchange, or decanting.

In some embodiments, the methods as described herein, include removing the particles from the second liquid through centrifugation, sieving, filtration, magnetic collection, solvent exchange, inertial separation, hydrocyclone separation, or decanting.

In other embodiments, the methods as described herein, further comprises washing the particles after step d) with a washing fluid, e.g., an organic liquid, a supercritical fluid, a cryogenic liquid, or a combination thereof. In certain embodiments, the washing fluid is an organic liquid, a supercritical fluid, a cryogenic liquid, or a combination thereof.

In certain embodiments, the methods further include washing the particles with a third liquid. In certain other embodiments, the third liquid is an organic solvent. The third liquid may also be removed through evaporation, vacuum desiccation or lyophilization, e.g., vacuum drying, with or without the application of heat, lyophilization, fluidized bed drying, tray drying, belt drying, or slurry spray drying. In still other embodiments, the particles are further dried by lyophilization or vacuum desiccation.

The drying of the particles, e.g., removal of the first and second liquids to produce dry particles, can be performed through methods as described herein. These include, but are not limited to, warm gas evaporation, freeze drying, critical point drying, emulsion solvent evaporation, emulsion solvent diffusion, or a combination thereof. In certain embodiments, the particles are further dried by lyophilization or vacuum desiccation.

In some embodiments, secondary desiccation is achieved by flowing a drying gas over a bed of particles atop a filtration element. In certain embodiments, the drying gas is helium, air, nitrogen or argon. In preferred embodiments, the drying gas is helium or air. The temperature, pressure, flow rate, or vapor content of the drying gas may be controlled during the drying time to achieve a desired rate of desiccation, a desired temperature difference relative to the glass transition temperature, or a desired equilibrium content of the first liquid or the second liquid at the conclusion of the secondary desiccation step. In other embodiments, the time required to achieve a desired level of desiccation is lower than that which corresponds to alternative secondary desiccation techniques, e.g., lyophilization, spray drying, or fluidized bed drying. Similarly, the percentage of material recovery may be greater.

In other embodiments, warm gas evaporation is used to further dry the particles. In some embodiments, the particles are further dried by contacting the particles with a stream of gas. In certain embodiments, the gas has a temperature from about −80 to about 200° C. In certain other embodiments, the gas has a temperature from about 10 to about 40° C. In still other embodiments, the gas has a relative humidity from greater than about 0% to less than about 100%. In certain preferred embodiments, the gas comprises helium, air, nitrogen or argon.

Sterility is a critical facet of pharmaceutical compositions because it affects the safety with which the composition may be administered. For example, many particle formulations, particularly microparticle formulations, achieving sterility can be a challenge since common sterilization techniques, e.g., sterile filtration, are not compatible with peptides. Sterile filtration steps typically involve a membrane through which only those components of the filtered liquid which are, for example, 200 nm in size or smaller may pass. Particle formulations with solids greater than 200 nm in size are therefore filtered rather than sterilized. In some embodiments, formulations of the disclosure are subjected to an alternative process of terminal sterilization prior to use or administration. The effectiveness of these sterilization protocols and of the process in reducing bioburden may be assessed following regulatory guidelines, e.g., those listed in USP Chapter <71>, Ph. Eur. Chapter, Sterility: 2.6.1, 21 CFR 610.12, ICH Q4B ANNEX 8(R1), ICH Q5A, etc. Exemplary methods of demonstrating compliance include incubating about 1 mL of the drug product per container in an appropriate growth media (Soybean—Casein Digest Medium, Tryptic Soy Broth, Fluid Thioglycollate Medium) for a period of about 14 days to ensure no microbial growth in about 1 in about 1000 million units of the drug product, or about 1 in about 1 million units of the drug product. As disclosed herein, a “sterile” formulation is aseptic or free from living microorganisms and their spores.

In some embodiments, the sterilization step involves gamma irradiation. In other embodiments, the sterilization step required to inactivate at least about 2-4 log₁₀ of viral microbial contaminants is about 10 kGy, about 20 kGy, about 40 kGy, about 60 kGy, or about 100 kGy. In certain embodiments, the particles comprise an antioxidant or a scavenger to mitigate the harmful effects of any degradation products which are generated as a result of the sterilization step.

In other embodiments, the sterilization step involves a transient thermal treatment. In some embodiments, the formulation is exposed to temperatures from about 60 to about 200° C., e.g., from about 60 to about 180° C., from about 60 to about 160° C., from about 60 to about 140° C., from about 60 to about 130° C., from about 60 to about 120° C., or from about 60 to about 110° C. In certain embodiments, the exposure occurs over a period from about 1 to about 144 hours, e.g., from about 1 to about 120 hours, from about 1 to about 100 hours, from about 1 to about 90 hours, from about 1 to about 72 hours, from about 1 to about 48 hours, from about 1 to about 36 hours, or from about 1 to about 24 hours. For example, dry heat sterilization can be performed at a temperature of about 80° C. for about 72 hours, about 160° C. for about two hours, or about 170° C. for about one hour. In certain other embodiments, pasteurization is performed at about 60° C. for about 10 hours.

In some embodiments, the sterilization is ensured by using beta radiation, X-ray sterilization, steam sterilization, solvent-detergent inactivation steps, supercritical CO₂ mediated sterilization, low pH holds, ultraviolet C exposure, or ethylene oxide mediated sterilization of the formulation. In other embodiments, the terminal sterilization step is performed at low temperatures from about −100 to about 60° C. In certain embodiments, the supercritical CO₂ further includes additives (e.g., hydrogen peroxide, water, acetic anhydride, etc.) intended to effectively inactivate microorganisms, including bacterial spores.

In other embodiments, the second liquid is chosen such that its presence in the drop during particle formation helps to facilitate process sterility. In some embodiments, the second liquid is an antimicrobial or contains such a compound which is contained within the particle. This compound may persist inside the particles even after a secondary drying step. Organic liquids that can be used as a second liquid with antimicrobial activity may include, but are not limited to acetates (e.g., ethyl acetate) and alcohols (e.g., ethanol, phenol), or the like. The second liquid may also contain antimicrobial excipients, e.g., phenolic substances, benzalkonium chloride, linalool, coumarin, peroxides, active chlorine, alkalis, or a combination thereof.

In some embodiments, the use of nano-filtration membranes for the inlet process streams, e.g., for use on the first liquid and/or the second liquid prior to particle formation, contributes to a reduction of the bio-burden on the process. In other embodiments, combinations of the preceding sterility measures are employed to reach appropriate bio-burden levels.

As described herein, the particles may be sterilized after formation, e.g., by irradiation, pasteurization, freezing, or irradiation by gamma radiation. In certain embodiments, the methods as described herein, further comprises sterilization of the particles after the first and second liquids are removed. In certain preferred embodiments, the sterilization occurs by irradiation, pasteurization, or freezing. In preferred embodiments, the irradiation is by gamma radiation.

The pharmaceutical compositions including suspensions or dry forms of the disclosure may be administered in a suitable dosage that may be adjusted as required, depending on the clinical response. Compositions may also be used cosmetically. The dosage of the pharmaceutical composition can vary depending on factors, such as the pharmacokinetics of the biologically active peptides; the mode of administration; the age, health, and weight of the recipient; the nature and extent of the symptoms; the frequency of the treatment, and the type of concurrent treatment, if any; and the clearance rate of the biologically active peptides in the animal to be treated. Administration may occur daily, weekly, every two weeks, every three weeks, monthly, or any other suitable interval.

The pharmaceutical composition may be administered by any suitable method, for example, by auricular, buccal, conjunctival, cutaneous, dental, electro-osmotical, endocervical, endosinusial, endotracheal, enteral, epidural, extra-amniotical, extracorporeal, infiltration, interstitial, intra-abdominal, intra-amniotical, intra-arterial, intra-articular, intrabiliary, intrabronchial, intrabursal, intracameral, intracardial, intracartilaginous, intracaudal, intracavernous, intracavitary, intracerebral, intracisternal, intracorneal, intracoronal, intracoronary, intracorporus cavernosum, intradermal, intradiscal, intraductal, intraduodenal, intradural, intraepidermal, intraesophageal, intragastrical, intragingival, intraileal, intralesional, intraluminal, intralymphatical, intramedullar, intrameningeal, intramuscular, intraocular, intraovarian, intrapericardial, intraperitoneal, intrapleural, intraprostatical, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastrical, occlusive dressing technique, ophthalmical, oral, oropharyngeal, parenteral, percutaneous, periarticular, peridural, perineural, periodontal, rectal, inhalation, retrobulbar, soft tissue, subarachnoidial, subconjunctival, subcutaneous, sublingual, submucosal, topical, transdermal, transmucosal, transplacental, transtracheal, transtympanic, ureteral, urethral, or vaginal administration.

The disclosure generically described, will be more readily understood by reference to the following examples, which are included merely for purposes of illustration of certain aspects and embodiments of the present disclosure, and are not intended to be limiting.

### EXEMPLIFICATION

### Abbreviations

Å angstrom
aa amino acids
BA butylacetate
agg aggregation
AU arbitrary units
BIgG bovine IgG
BSA bovine serum albumin
° C. degrees Celsius
cm centimeter
conc. concentration
cP centipoise
CP continuous phase
d day
DCM dichloromethane
DI deionized
DIPEA diisopropylethylamine
DMA N,N-dimethylaniline
DMF dimethyl formamide
DMSO dimethyl sulfoxide
DP dispersed phase
DTE dithioerythritol
DTT dithiothreitol
EDT 1,2-ethanedithiol
EDTA ethylenediaminetetraacetic acid
EO ethyloleate
eq. equivalent
Et ethyl
eV electron-volts
g gram
h hour

HIgG human IgG
HPLC high performance liquid chromatography
hr hour
HSA human serum albumin
Hz hertz
ID internal diameter
IV intravenous
KF Karl Fischer
kJ kilojoules
kPa kiloPascal
kV kilovolts
LC-MS liquid chromatograph mass spectrometry
L liter
m meta
mAb monoclonal antibody
MALDI-MS matrix-assisted laser desorption ionization mass spectrometry
Me methyl
MHz megahertz
min minute
microgram
microliter
μm micrometer
μM micromolar
mg milligram
mL milliliter
mm millimeter
mM millimolar
mol mole
mPa·s milliPascal second
mTorr milliTorr
N newton
nBA n-butylacetate
nm nanometer
NMP N-methylpyrrolidone
p para
PBS phosphate-buffered saline
PEG polyethylene glycol
PEGA polyethylene glycol polyacrylamide
PI pressure indicator
ppm parts per million
ps picosecond
PTFE polytetrafluoroethylene
rcf relative centrifugal force
RH relative humidity
RP-HPLC reversed phase-high performance liquid chromatography
rpm revolutions per minute
RT room temperature
s second
SC subcutaneous
sec second
SEM scanning electron microscopy
SVP subvisible particle
t tertiary
tert tertiary
UHMW ultrahigh molecular weight polyethylene
ug micrometer
UTW ultra thin wall
UOM unit of measure
UV ultraviolet
V volts
vol % volume percent
v/v volume per volume
W watt
wt % weight percent
w/v weight per volume
w/w weight per weight
wt/wt weight per weight

### Materials

Peptides were purchased from MyTide Therapeutics (Boston, Mass.) and received as a solid. Composition of custom “feed solutions” used for processing particles were produced through modifying the FDA-label formulation by desalting followed by concentrating and adding desired excipients. Concentration columns were procured from Millipore Sigma (Amicon® Ultra 15 mL Filters for Protein Purification and Concentration with a 3 kDa cut off) and used where necessary to: (i) reach the desired peptide concentration, and (ii) exchange buffer/excipients before particle formation. Zeba desalting columns (Thermo Fisher Scientific 87773) were also used to remove salt from solutions in certain instances. Typically, the ratio of residual salt to peptide in the desalted solutions (wt/wt) was <1%. All excipients were purchased from Sigma-Aldrich and used as received.

Desiccation liquids, i.e., second liquids, including benzyl benzoate, various alcohols, various acetates, oils, ionic liquids, surfactants, and aqueous media comprising different forms of polyethylene glycol (PEG) were used as appropriate. Benzyl benzoate is an organic liquid, largely immiscible with water, which exhibits a density (d=1.12 g/cm³) that typically brackets that of the liquid feed solution (d≠1 g/cm³ in the case of water) and the density of solid proteins, i.e., the density of the dry protein powder (d≠1.25-1.35 g/cm³). It therefore served as a medium upon which drops floated while undergoing primary desiccation via dispersal of the first liquid in the benzyl benzoate and evaporation of the first liquid in the surrounding medium, e.g., air (typically of order several seconds or less). The desiccated particles sunk thereafter, such that a spatial separation was generated between wet incoming drops and processed particles. Such separation helped to mitigate particle coalescence, among other phenomena. The remaining liquids typically exhibited a density less than or approximately that of the feed solution. Drops did not tend to float, and primary desiccation was therefore driven primarily by dispersal of the first liquid in the second liquid. All desiccation (“second”) liquids, e.g., acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, except for the ionic liquids were purchased from Sigma Aldrich and used as received. The ionic liquids were purchased from TCI America and used as received.

### Methods

Particle Formation: Unless otherwise noted, an electrospray apparatus, an ultrasonic atomizer, vortex, or a microfluidic device was used to form drops for desiccation and particle formation. In most instances the electrospray apparatus comprised a Sono-Tek 120 kHz ultrasonic atomizer charged by a Matsusada EQ-30P1-LCt or EQ-30N1-LCt high voltage DC power supply, while in others it was replaced by a small blunt disposable syringe needle (VWR International). A Harvard Apparatus Model 33 dual-channel syringe pump was utilized for pumping the feed solution. The needle was charged by a Matsusada EQ-30P1-LCtG high voltage DC power supply. For select samples, the needle was replaced by a Sono-Tek 120 kHz ultrasonic atomizer nozzle driven at a power of 4.5 W. The drops generated by the apparatus were collected for desiccation by a vessel containing the second liquid, typically under conditions of continuous stirring. Thermal management of the second liquid was utilized in the preparation of select samples. The distance between the surface of the second liquid in the vessel and the tip of the drop source was typically 10-20 cm.

The feed solution was diluted to reach a final concentration of 25 mg/mL. 25mL of the second liquid was added to a 50 mL conical tube. An 80 μL feed was then added to each tube. The tube was immediately vortexed for 30 seconds at the maximum rate possible (speed=10) using the Vortex Genie vortexer. For acetates, droplets were dehydrated completely within 30 seconds. The exact dehydration time depended upon the solvent used. To isolate protein microspheres from the first and second liquids, tubes were centrifuged at 100 g for 2 minutes. Supernatant was removed by a quick inversion into a waste collector. The remaining solids were resuspended in less than 1 mL of the second liquid and transferred to a 1.5 mL centrifuge tube. The 1.5 mL tubes were centrifuged at 100 g for 2 minutes and excess liquids were removed using a pipette. Removal of the residual liquids was accomplished by vacuum drying for 2 hours. The solids were optionally re-dissolved in DI water by gentle rocking on a platform shaker for at least 15 minutes and analyzed further for aggregation using turbidity, FlowCam and SEC analysis.

Lyophilization: The particles for lyophilized samples, i.e., samples marked as having gone through a secondary desiccation step involving freeze drying, were loaded into either microcentrifuge or 15 mL conical tubes and subjected to snap freezing by immersion in liquid nitrogen for approximately 10 min. The samples were then loosely covered and transferred to either a Virtis Advantage or a Labconco FreeZone lyophilizer for approximately 24 hours at a pressure of approximately 10-50 mTorr.

FlowCam: Particle sizing was measured using FlowCam; a dynamic image analysis instrument. Samples were diluted to about 1 mg/mL in isopropanol and passed through a thin channel. Images of particles were recorded and analyzed according to size and shape.

ImageJ Measurements: Particles diameters were measured using ImageJ analysis on SEM images. The analysis was performed on the 600× images. The ImageJ Particle Analysis tool was run on the image, identifying objects with a circularity of >0.8 and size >0.5 μm with each object outlines. These outlines were visually inspected for good fit. Any mis-identified particles were manually rejected and any missed particles were manually included and measured using the ImageJ diameter tool.

Accelerated Storage Protocol: All samples were transferred to Wheaton E-Z extraction round-bottom glass vials for aging (2 mL or 4 mL volume, depending on sample). The glass vials were sealed with parafilm, placed in an oven at 40° C., and visually inspected on a daily basis over the aging period to ensure integrity and stability.

Viscosity Measurements: Apparent suspension viscosity was measured using an AR-G2 rheometer (TA Instruments) and a 25 mm plate at 25° C. Measurements were taken at 1000 s−1 (experimental limit due to edge effects), which is below the shear rates experienced in 27-gauge needles, but in the Newtonian regime for the suspensions. Each measurement was repeated three times (about 60 s intervals between repeats) to assess short-term physical stability of the suspensions. Prior to each measurement calibration standards were recorded to validate instrument settings.

Karl Fischer: Testing for moisture content was undertaken using Karl Fischer analysis. Approximately 100 mg of particles was heated to 105° C. in an oven and released water was determined coulometrically.

Skeletal Density: Skeletal density was measured by gas pycnometry. The gas was nitrogen or helium and the particle mass was about 0.04 g to about 0.6 g at temperatures at about 22° C.

Particle Dissolution: Phosphate-buffered saline (PBS) was added to dry particle samples to produce a final concentration of 10 mg/mL (particle mass/mL of solution). Samples were placed on a VWR angular rocker with a speed setting of “35” and angle setting of “15”. At 1, 10, 20, 30, 40, 50 ,60, 90, and 120 minutes a 10 μL aliquot was removed from the sample vial and the absorbance at 280 nm was measured and recorded. The peptide concentration was plotted against time for all samples.

Salt Content: Salt content was recorded by measuring sodium content using Inductively Coupled Plasma Optical Emission Spectroscopy (ICP-OES). A calibration curve was prepared using a sodium standard (ICPTraceCERT, 1000 mg/L). Quality control was completed using a diluted standard solution at 100 ppm sodium. A sample of particles (˜15 mg) dissolved in 2 vol % nitric acid (10 mL) was then analyzed, resulting in an intensity lower than the instrument detection limit of ˜0.5 ppm for sodium. This indicated a sodium content of less than 0.034 wt % and a total salt content (assuming sodium citrate and sodium chloride to have been removed equally) of less than 0.1 wt %.

Size Exclusion Chromatography (SEC) Measurements: The quantification of size variants in select samples was determined by size exclusion chromatography. The analysis utilized an AdvanceBio SEC-3 column, 7.8 mm ID×30 cm, 3 μm (Agilent) run on an HPLC system (1260 Infinity II, Agilent). The mobile phases were 25 mM potassium phosphate and 0.25 M potassium chloride at pH 6.8. The chromatography was run isocractically at a flow rate of 1.0 mL/min for 15 minutes. The column temperature was maintained at ambient 25° C. and the eluent absorbance was monitored at 280 nm. Each monoclonal antibody was diluted with its respective formulation buffer to 1 mg/mL. The injection volume was 10 μL. 20 μL Injections of samples (1 mg/mL) were run at a flow rate of 1 mL/min in SEC buffer (25 mM phosphate, 250 mM NaCl pH 6.8) for 15 minutes on an Agilent AdvanceBio SEC (300 mm×2.7 μm, 300 Å column). Peak analysis was performed by auto-integrating using the following parameters: slope sensitivity=0.5, peak width=0, height reject=0, area reject=0, shoulders off, area percent reject 0, standard tangent skim mode, advanced baseline correction, 0 for front peak skim height ratio, 0 for tail peak skim height ratio, 0 for peak to valley ratio, and 0 for skim valley ratio.

Differential Scanning Fluorimetry (DSF) Measurements: The melting temperature of the protein before and after formulation, as well as at various time points of 40° C. storage, were assessed using a QuantStudio 6 Flex instrument. Five microliters (5 μL) of samples (1 mg/mL), prepared after dialysis, were loaded onto a 96-well thermal cycler plate in quadruplicate. To each well, 12.5 μL of ultrapure deionized water and 2.5 μL of SYPRO® Orange dye (8×) were added. After a 5-minute incubation, samples were run from 25° C. to 99° C. at a ramp rate of 0.05° C./s. Melting temperature was calculated using the Protein Thermal Shift Software (Thermo Fisher, version 1.3) using a Boltzmann fit.

Circular Dichroism (CD) Measurements: The degree of preservation of the secondary structure (alpha helices and beta sheets) of the protein before and after formulation, as well as at various time points of 40° C. storage, was assessed using a Jasco J-815 instrument. Four hundred microliters (400 μL) of sample (0.5 mg/mL), prepared after dialysis, was loaded into a quartz cuvette (1 mm path length). Samples were scanned over the 190-260 nm range. Diluent buffer was used as blank subtraction for each sample. The following instrument settings were used:

Photometric mode: CD, HT

Measure range: 260-190 nm

Data pitch: 0.5 nm

Sensitivity: Standard

D.I.T.: 4 sec

Bandwidth: 1.00 nm

Start mode: Immediately

Scanning speed: 100 nm/min

Shutter control: Auto

Baseline correction: None

CD detector: PMT

PMT voltage: Auto

Cation Exchange Chromatography (CIEX) Measurements: Charge variant analysis was performed for each sample on days 0, 7 and 30 under accelerated storage conditions, using an Agilent BioMAb NPS, 4.6×250 mm, PEEK ion exchange column. Samples were prepared at 1 mg/mL concentration after overnight dialysis in water. Buffer A was prepared with: 30 mM phosphate, pH: 6.3, and NaCl: 0 mM. Buffer B was prepared with: Buffer A: 30 mM phosphate, pH 6.3 plus NaCl: 175 mM. The samples were run in a gradient starting with 100% Buffer A, ramping up to a 100% Buffer B over a course of 20 min, after which the gradient was set to return to 100% Buffer A and 0% Buffer B in the next 1 min. The system re-equilibrated in 100% Buffer A for 10 min before the injection of the next sample. Integration was performed as a manual skim peak mode to reflect the Agilent data in the following protocol: https://www.agilent.com/cs/library/applications/5991-5557EN.pdf.

Scanning Electron Microscopy (SEM): Electron micrographs were collected for select samples with either a Hitachi TM3030Plus or a TM1000 tabletop microscope. The samples were immobilized on conductive tape and examined in a low-vacuum anti-charging environment, obviating the need for sample preparation.

Image Analysis: Select microscopy images were chosen for further analysis on the basis of (i) minimal particle overlapping, (ii) good contrast between the particles and the background, and (iii) a resolution providing for particle occupancies of at least 10 pixels. This allowed for particles to be easily identified and reduced resolution-based error. A binary threshold was applied to separate the particles from background, and a watershed segmentation algorithm was applied to ensure that individual particles were measured separately. The ImageJ tool “Analyze Particles” was then applied on the binary picture with the following parameters: circularity between 0.5 and 1.0; size between 5 and infinity square microns; exclude on edges; fill holes. The outlines of the identified particles were overlaid onto the original image. Particles which were misidentified, such as clusters that were identified as a single particle or particles whose outlines do not match the particle, were then discarded. Missing particles were measured by manually tracing the particle's outline and using ImageJ's Measure tool.

Density Analysis: The skeletal density of particles from select samples was determined by examining approximately 0.1 g of powder with an AccuPyc II 1340 gas displacement pycnometry system.

Water Content Analysis: The residual moisture in particles from select samples was determined by placing approximately 0.1 g of powder in an oven with a Karl Fischer titrator and heating the sample.

Accelerated Storage: Storage was carried out under accelerated conditions for select samples by maintaining them at an elevated temperature (40° C.) for defined periods of time in an incubator or oven. Samples were kept in 2 mL or 4 mL Wheaton glass vials and sealed with paraffin film.

Helium Ion Microscopy (HIM): Ion micrographs were collected for select samples using an HIM instrument. The source energy, working distance, and aperture size were typically, 29 keV, 9 mm, and 10 microns, respectively. For select samples, a focused gallium ion beam was used to section particles for analysis of the internal structure. Tilted samples were ablated with a source current, dwell time, and cut spacing of 300 pA, 0.5-1 μs, and 2-5 nm, respectively.

X-Ray Photoelectron Spectroscopy (XPS): A small amount of powder was deposited onto hydrocarbon tape attached to a piece of silicon wafer and gently pressed to form a compact uniform bed. Excess loose powder was removed by lightly tapping the edge of the wafer piece. Specimens were prepared just before analysis. XPS measurements were performed with a Kratos Axis Ultra spectrometer using monochromatic Al Kα X-rays (1486.6 eV). For each sample, a survey spectrum was acquired from an area of approximately 2 mm by 1 mm (pass energy=160 eV; 225W power), from which the surface elemental composition was determined. Charge compensation was achieved using a beam of magnetically focused electrons as a flood current. The standard photoelectron take-off angle used for analysis is 90° giving a sampling depth in the range 5-8 nm. The surface elemental compositions were analyzed using a quantification model that assumes homogeneity of the probed sample volume.

Inverse Gas Chromatography (IGC): Powdered samples were analyzed using inverse gas chromatography. Cylindrical columns were packed with 200 to 300 mg of powdered samples to make up a stationary phase. Following an inert gas purge, a series of gas probes was injected on the column. Determination of the retention volume for each probe enabled evaluation of the dispersive and polar components of the surface energy for each sample.

X-Ray Diffraction (XRD): Samples were packed into 0.7 mm diameter glass capillaries. The powder patterns were measured on a PANalytical Empyrean diffractometer equipped with an incident-beam focusing mirror and an X'Celerator detector. The patterns (1-50° 2θ, 0.0167113° steps, 4 sec/step, 1/4° divergence slit, 0.02 radian Soller slits) were measured using Mo Ká radiation. If static electricity effects (for the case of evaluating a lyophilization control this occurred after grinding in a mortar and pestle) prevented packing the sample into a capillary, its powder pattern was measured from a flat plate specimen on a Bruker D2 Phase diffractometer equipped with a LynxEye position-sensitive detector. The pattern was measured using Cu Kα radiation from 5-100° 2θ in 0.0202144° steps, counting for 1.0 sec/step. The standard instrument settings (30 kV, 10 mA, 0.6 mm divergence slit, 2.5° Soller slits, and 3 mm scatter screen height) were employed.

Microflow Particle Sizing (MPS): Flow imaging microscopy for particle size analysis was carried out using a FlowCam PV-100. To investigate size and dispersity of particles, 5 mg of powder were dispersed in 10 mL of dry isopropanol via sonication. The isopropanol continuous phase prevented the particles from dissolving, i.e., prevented reconstitution. 0.3 mL was injected into the cell and images of the particles were taken using a flow rate of 0.15 mL/minute. Particles with a circularity greater than 0.9 were reported in the analysis and any double images were removed from the analysis, to give a size distribution and dispersity of particles in the range from 1 to 100 μm.

Dynamic Vapor Sorption (DVS): Powders were analyzed using dynamic water vapor sorption. Approximately 50 mg of powdered sample was loaded into the pan of the instrument's microbalance. The sample was held isothermally at 22° and the sample mass was monitored throughout the measurement. Following a 0% RH purge to remove surface water, the relative humidity (RH) in the sample chamber was ramped at a constant rate of 4% RH per hour up to 90% RH. The sample was held at 90% RH for one hour, then the RH was reduced to 0% as a step change. The sample was held at 0% RH for one hour, after which the measurement was terminated.

Dynamic Scanning calorimetry (DSC): Powdered samples were analyzed using dynamic scanning calorimetry. Masses of 5 to 10 mg of powdered samples were loaded into aluminum crucibles and sealed hermetically. Crucibles were loaded into the instrument, and the heat flow into the samples was monitored while the temperature was ramped from 30 to 250° C. at a constant rate of 5° C./minute.

USP <790>: According to the USP <790> standard, samples of dissolved particles were visually observed against a white and black background under lighting conditions greater than 2000 lux. Matte-finished high density polyethylene sheets were selected for the background to reduce glare. The illuminance at the viewing point was confirmed with a lux meter (Dr. Meter, LX1330B). The samples were swirled before being held up to the backgrounds and viewed for 5 sec.

### EXAMPLES

The methods disclosed herein, have been utilized in separate instances to prepare particles including at least one of several peptides. Various analytical techniques were applied to assess the physical characteristics of the particles themselves as well as the structural and functional properties of the processed peptides. Scanning electron microscopy and associated image analysis were used to study the particle morphology and size distribution, respectively. Various morphologies and distributions of components were achieved by controlling the properties of the first liquid and/or the second liquid. In some instances, the processing conditions conferred smooth particles of high sphericity and/or facile control of the mean particle size over a broad range with low dispersity. In certain cases, the particle surfaces were also decorated with components, e.g., excipients, in a controlled fashion. Density and water content measurement demonstrated that the particles approached crystalline packing efficiencies and retained very low levels of residual moisture after post-processing. Finally, investigation of the insoluble particle populations upon reconstitution revealed very few insoluble artifacts, particularly as compared to alternative particle formation procedures.

### Example 1

The following solutions were used as the feed solution (dispersed phase) for the standard methods:

1. 3 mg/mL peptide in 30 mg/mL Trehalose and 0.1% (w/v) PS80

2. 3 mg/mL peptide in 30 mg/mL Arginine.HCl and 0.1% (w/v) PS80

3. 3 mg/mL peptide in 15 mg/mL Trehalose, 15 mg/mL Arginine.HCl and 0.1% (w/v) PS80

### Example 2

Vortex process: The dispersed phase (DP) was 80 μL of the feed prepared at 3 mg/mL peptide in 30 mg/mL Trehalose and 0.1% (w/v) PS80. The continuous phase (CP) was 25 mL of n-butyl acetate. The DP and CP were mixed by vortexing for 30 s. This mixture was collected in a beaker with droplets dehydrating and forming particles. These particles were filtered and collected. SEM images revealed identifiable circular particulate matter.

### Example 3

Vortex process: The dispersed phase (DP) was 80 μL of the feed prepared at 3 mg/mL peptide in 30 mg/mL Arginine.HCl and 0.1% (w/v) PS80. The continuous phase (CP) was 25 mL of n-butyl acetate. The DP and CP were mixed by vortexing for 30 s. This mixture was collected in a beaker with droplets dehydrating and forming particles. These particles were filtered and collected. SEM images revealed identifiable circular particulate matter.

### Example 4

Vortex process: The dispersed phase (DP) was 80 μL of the feed prepared at 3 mg/mL peptide in 15 mg/mL Trehalose, 15 mg/mL Arginine.HCl and 0.1% (w/v) PS80. The continuous phase (CP) was 25 mL of n-butyl acetate. The DP and CP were mixed by vortexing for 30 s. This mixture was collected in a beaker with droplets dehydrating and forming particles. These particles were filtered and collected. SEM images revealed identifiable circular particulate matter.

### Example 5

Vortex process: The dispersed phase (DP) was 20 μL of the feed prepared at 3 mg/mL peptide in 30 mg/mL Trehalose and 0.1% (w/v) PS80. The continuous phase (CP) was 25 mL of n-butyl acetate. The DP and CP were mixed by vortexing for 30 s. This mixture was collected in a beaker with droplets dehydrating and forming particles. These particles were filtered and collected. SEM images revealed identifiable circular particulate matter.

### Example 6

Vortex process: The dispersed phase (DP) was 40 μL of the feed prepared at 3 mg/mL peptide in 30 mg/mL Trehalose and 0.1% (w/v) PS80. The continuous phase (CP) was 25 mL of n-butyl acetate. The DP and CP were mixed by vortexing for 30 s. This mixture was collected in a beaker with droplets dehydrating and forming particles. These particles were filtered and collected. SEM images revealed identifiable circular particulate matter.

### Example 7

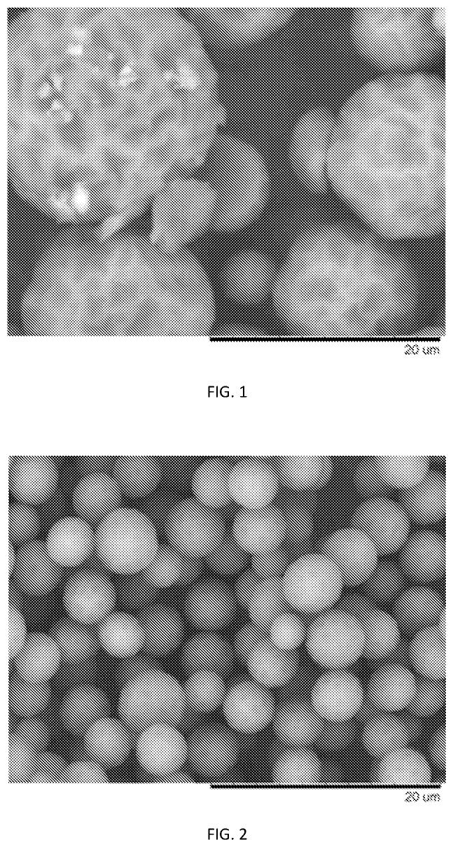

Vortex process: The dispersed phase (DP) was 40 μL of the feed prepared at 3 mg/mL peptide in 30 mg/mL Trehalose and 0.1% (w/v) PS80. The continuous phase (CP) was 25 mL of n-butyl acetate. The DP and CP were mixed by vortexing for 300 s. This mixture was collected in a beaker with droplets dehydrating and forming particles. These particles were filtered and collected. SEM images revealed circular particulate matter containing 8.8% (w/w) peptide, 88.2% (w/w) trehalose and 3% (w/w) PS80 at 20 μm as shown in FIG. 1.

### Example 8

Ultrasonic atomizer process: The dispersed phase (DP) was 480 μL of the feed prepared at 3 mg/mL peptide in 30 mg/mL Trehalose and 0.1% (w/v) PS80. The continuous phase (CP) was 300 mL of n-butyl acetate. Inlet feed flow rate=0.05 mL/min. The electric field was −10 kV. The DP and CP were mixed by vortexing for 30 s. This mixture was collected in a beaker with droplets dehydrating and forming particles. These particles were filtered and collected. SEM images revealed circular particulate matter containing 8.8% (w/w) peptide, 88.2% (w/w) trehalose and 3% (w/w) PS80 at 20 μm as shown in FIG. 2. The average circularity was calculated to be 0.87, standard deviation 0.013.

### INCORPORATION BY REFERENCE

All publications and patents mentioned herein, are hereby incorporated by reference in their entirety as if each individual publication or patent was specifically and individually indicated to be incorporated by reference. In case of conflict, the present application, including any definitions herein, will control.

### EQUIVALENTS

While specific aspects and embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A particle comprising a peptide, wherein the particle comprises up to about 10% (w/w) of peptide and the circularity of the particle is from about 0.10 to about 1.00.

2. The particle of claim 1, wherein the peptide is a biologically active peptide.

3. The particle of claim 2, wherein the biologically active peptide comprises from about 2 to about 12 amino acid residues.

4. The particle of any one of the preceding claims, wherein the particle comprises up to about 8% (w/w) of peptide.

5. The particle of claim 4, wherein the particle comprises up to about 6% (w/w) of peptide.

6. The particle of claim 4 or 5, wherein the particle comprises up to about 5% (w/w) of peptide.

7. The particle of any one of claims 4-6, wherein the particle comprises up to about 3% (w/w) of peptide.

8. The particle of any one of the preceding claims, wherein the circularity of the particle is from about 0.80 to about 1.00.

9. The particle of claim 8, wherein the circularity of the particle is from about 0.85 to about 1.00.

10. The particle of claim 8 or 9, wherein the circularity of the particle is from about 0.90 to about 1.00.

11. The particle of any one of claims 8-10, wherein the circularity of the particle is from about 0.95 to about 1.00.

12. The particle of any one of claims 8-11, wherein the circularity of the particle is from about 0.98 to about 1.00.

13. The particle of any one of claims 8-12, wherein the circularity of the particle is about

14. The particle of any one of the preceding claims, wherein the particle has a substantially smooth surface.

15. The particle of any one of the preceding claims, wherein the particle has a diameter of about 0.1 to about 100 μm.

16. The particle of claim 15, wherein the particle has a diameter of about 1 to about 100 μm.

17. The particle of claim 15 or 16, wherein the particle has a diameter of about 5 to about 100 μm.

18. The particle of any one of claims 15-17, wherein the particle has a diameter of about 5 to about 50 μm.

19. The particle of any one of claims 15-18, wherein the particle has a diameter of about 5 to about 20 μm.

20. The particle of any one of the preceding claims, wherein the particle has a skeletal density of about 1.00 to about 6.00 g/cm³.

21. The particle of claim 20, wherein the particle has a skeletal density of about 1.15 to about 1.60 g/cm³.

22. The particle of claim 20 or 21, wherein the particle has a skeletal density of about 1.25 to about 1.50 g/cm³.

23. The particle of any one of claims 20-22, wherein the particle has a skeletal density of about 1.30 to about 1.40 g/cm³.

24. The particle of any one of the preceding claims, wherein the particle has a glass transition temperature that is higher than about 60° C.

25. The particle of claim 24, wherein the particle has a glass transition temperature that is higher than about 90° C.

26. The particle of claim 24 or 25, wherein the particle has a glass transition temperature that is higher than about 100° C.

27. The particle of any one of claims 24-26, wherein the particle has a glass transition temperature that is higher than about 130° C.

28. The particle of any one of claims 24-27, wherein the particle has a glass transition temperature that is higher than about 170° C.

29. The particle of any one of the preceding claims, further comprising a carbohydrate, a protein stabilizer, an emulsifier, an amino acid, a surfactant, or a combination thereof.

30. The particle of claim 29, wherein the carbohydrate is dextran, trehalose, sucrose, agarose, mannitol, lactose, sorbitol, maltose, or a combination thereof.

31. The particle of claim 29, wherein the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, or a combination thereof.

32. The particle of claim 29, wherein the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

33. The particle of claim 29, wherein the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

34. The particle of claim 29, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof.

35. The particle of any one of the preceding claims, wherein the particle has a surfactant content of less than about 10% by mass.

36. The particle of claim 35, wherein the particle has a surfactant content of less than about 5% by mass.

37. The particle of claim 35 or 36, wherein the particle has a surfactant content of less than about 3% by mass.

38. The particle of any one of claims 35-37, wherein the particle has a surfactant content of less than about 1% by mass.

39. The particle of any one of claims 35-38, wherein the particle has a surfactant content of less than about 0.1% by mass.

40. The particle of any one of claims 35-39, wherein the particle is substantially free from any surfactant content.

41. The particle of any one of the preceding claims, wherein the particle has less than about 7% residual moisture by weight.

42. The particle of claim 41, wherein the particle has less than about 5% residual moisture by weight.

43. The particle of claim 41 or 42, wherein the particle has less than about 3% residual moisture by weight.

44. The particle of any one of claims 41-43, wherein the particle has less than about 1% residual moisture by weight.

45. A composition comprising a plurality of particles, each particle comprising a peptide suspended in a liquid, wherein the particles comprise up to about 10% (w/w) of peptide and the circularity of the particles is from about 0.10 to about 1.00.

46. The composition of claim 45, wherein the peptide is a biologically active peptide.

47. The composition of claim 46, wherein the biologically active peptide comprises from about 2 to about 12 amino acid residues.

48. The composition of any one of claims 45-47, wherein the particles comprise up to about 8% (w/w) of peptide.

49. The composition of claim 48, wherein the particles comprise up to about 6% (w/w) of peptide.

50. The composition of claim 48 or 49, wherein the particles comprise up to about 5% (w/w) of peptide.

51. The composition of any one of claims 48-50, wherein the particles comprise up to about 3% (w/w) of peptide.

52. The composition of any one of claims 45-51, wherein the circularity of the particles is from about 0.80 to about 1.00.

53. The composition of claim 52, wherein the circularity of the particles is from about

0. 85 to about 1.00.

54. The composition of claim 52 or 53, wherein the circularity of the particles is from about 0.90 to about 1.00.

55. The composition of any one of claims 52-54, wherein the circularity of the particles is from about 0.95 to about 1.00.

56. The composition of any one of claims 52-55, wherein the circularity of the particles is from about 0.98 to about 1.00.

57. The composition of any one of claims 52-56, wherein the circularity of the particles is about 1.00.

58. The composition of any one of claims 45-57, wherein the particles have a substantially smooth surface.

59. The composition of any one of claims 45-58, wherein the particles have a diameter of about 0.1 to about 100 μm.

60. The composition of claims 59, wherein the particles have a diameter of about 1 to about 100 μm.

61. The composition of any one of claims 59-60, wherein the particles have a diameter of about 5 to about 100 μm.

62. The composition of any one of claims 59-61, wherein the particles have a diameter of about 5 to about 50 μm.

63. The composition of any one of claims 59-62, wherein the particles have a diameter of about 5 to about 20 μm.

64. The composition of any one of claims 45-63, wherein the particles have a skeletal density of about 1.00 to about 6.00 g/cm³.

65. The composition of claim 64, wherein the particles have a skeletal density of about 1.15 to about 1.60 g/cm³.

66. The composition of claim 64 or 65, wherein the particles have a skeletal density of about 1.25 to about 1.50 g/cm³.

67. The composition of any one of claims 64-66, wherein the particles have a skeletal density of about 1.30 to about 1.40 g/cm³.

68. The composition of any one of claims 45-67, wherein the particles have a glass transition temperature that is higher than about 60° C.

69. The composition of claim 68, wherein the particles have a glass transition temperature that is higher than about 90° C.

70. The composition of claim 68 or 69, wherein the particles have a glass transition temperature that is higher than about 100° C.

71. The composition of any one of claims 68-70, wherein the particles have a glass transition temperature that is higher than about 130° C.

72. The composition of any one of claims 68-71, wherein the particles have a glass transition temperature that is higher than about 170° C.

73. The composition of any one of claims 45-72, wherein the particles further comprise a carbohydrate, a protein stabilizer, an emulsifier, an amino acid, a surfactant, or a combination thereof.

74. The composition of claim 73, wherein the carbohydrate is dextran, trehalose, sucrose, agarose, mannitol, lactose, sorbitol, maltose, or a combination thereof.

75. The composition of claim 73, wherein the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, or a combination thereof.

76. The composition of claim 73, wherein the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

77. The composition of claim 73, wherein the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

78. The composition of claim 73, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof.

79. The composition of any one of claims 45-78, wherein the particles have a surfactant content of less than about 10% by mass.

80. The composition of claim 79, wherein the particles have a surfactant content of less than about 5% by mass.

81. The composition of claim 79 or 80, wherein the particles have a surfactant content of less than about 3% by mass.

82. The composition of any one of claims 79-81, wherein the particles have a surfactant content of less than about 1% by mass.

83. The composition of any one of claims 79-82, wherein the particles have a surfactant content of less than about 0.1% by mass.

84. The composition of any one of claims 79-83, wherein the particles are substantially free from any surfactant content.

85. The composition of any one of claims 45-84, wherein the particles have less than about 7% residual moisture by weight.

86. The composition of claim 85, wherein the particles have less than about 5% residual moisture by weight.

87. The composition of claim 85 or 86, wherein the particles have less than about 3% residual moisture by weight.

88. The composition of any one of claims 85-87, wherein the particles have less than about 1% residual moisture by weight.

89. The composition of any one of claims 45-88, wherein the liquid is an organic solvent or ionic liquid.

90. The composition of claim 89, wherein the organic solvent comprises benzyl benzoate, coconut oil, cottonseed oil, fish oil, grape seed oil, hazelnut oil, hydrogenated vegetable oils, olive oil, palm seed oil, peanut oil, peppermint oil, safflower oil, sesame oil, soybean oil, sunflower oil, walnut oil, acetone, ethyl acetate, ethyl lactate, dimethylacetamide, dimethyl isosorbide, dimethyl sulfoxide, glycofurol, diglyme, methyl tert-butyl ether, N-methyl pyrrolidone, perfluorodecalin, polyethylene glycol, 2-pyrrolidone, tetrahydrofurfuryl alcohol, trigylcerides, triglycerides of the fractionated plant fatty acids C8 and C10, propylene glycol diesters of saturated plant fatty acids C8 and C10, ethyl oleate, ethyl caprate, dibutyl adipate, fatty acid esters, hexanoic acid, octanoic acid, triacetin, diethyl glycol monoether, gamma-butyrolactone, eugenol, clove bud oil, citral, limonene, polyoxyl 40 hydrogenated castor oil, polyoxyl 35 castor oil, simple alcohols such as ethanol, octanol, hexanol, decanol, propanol, and butanol, gamma-butyrolactone, tocopherol, octa-fluoropropane, (perfluorohexyl)octane, n-acetyltryptophan, ethyl laurate, methyl caprylate, methyl caprate, methyl myristate, methyl oleate, methyl linoleate, dimethyl adipate, dibutyl suberate, diethyl sebacate, ethyl macadamiate, trimethylolpropane triisosterate, isopropyl laurate, isopropyl myristate, diethyl succinate, polysorbate esters, ethanol amine, propanoic acid, citral, anisole, anethol, benzaldehyde, linalool, caprolactone, phenol, thioglycerol, dimethylacetamide, diethylene glycol monoethyl ether, propylene carbonate, solketal, isosorbide dimethyl ether, ethyl formate, and ethyl hexyl acetate, or a combination thereof.

91. The composition of claim 89 or 90, wherein the organic solvent is ethyl oleate, trigylcerides, ethyl laurate, ethyl macadamiate, ethyl caprate, diethyl succinate, diethylene glycol monoethyl ether, propylene carbonate, or a combination thereof.

92. The composition of any one of claims 89-91, wherein the organic solvent is ethyl oleate or trigylcerides.

93. The composition of claim 89, wherein the ionic liquid comprises pyridinium, pyridazinium, pyrimidinium, pyrazinium, imidazolium, pyrazolium, thiazolium, oxazolium, triazolium, ammonium, sulfonium, halides, sulfates, sulfonates, carbonates, phosphates, bicarbonates, nitrates, acetates, PF₆⁻, BF₄⁻, triflate, nonaflate, bis(trifyl)amide, trifluoroacetate, heptafluorobutanoate, haloaluminate, or a combination thereof.

94. The composition of any one of claims 45-93, wherein the liquid is a pharmaceutically acceptable liquid.

95. The composition of any one of claims 45-94, wherein the liquid further comprises a carbohydrate, a pH adjusting agent, a salt, a chelator, a mineral, a polymer, a surfactant, a protein stabilizer, an emulsifier, an antiseptic, an amino acid, an antioxidant, a protein, an organic solvent, a paraben, a bactericide, a fungicide, a vitamin, a preservative, a nutrient media, analgesic, or a combination thereof.

96. The composition of claim 95, wherein the carbohydrate is dextran, trehalose, sucrose, agarose, mannitol, lactose, sorbitol, maltose, or a combination thereof.

97. The composition of claim 95, wherein the pH adjusting agent is acetate, citrate, glutamate, glycinate, histidine, lactate, maleate, phosphate, succinate, tartrate, bicarbonate, aluminum hydroxide, phosphoric acid, hydrochloric acid, DL-lactic/glycolic acids, phosphorylethanolamine, tromethamine, imidazole, glyclyglycine, monosodium glutamate, sodium hydroxide, potassium hydroxide, or a combination thereof.

98. The composition of claim 95, wherein the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, magnesium chloride, potassium nitrate, or a combination thereof.

99. The composition of claim 95, wherein the chelator is disodium edetate, ethylenediaminetetraacetic acid or pentetic acid.

100. The composition of claim 95, wherein the mineral is calcium, zinc, or titanium dioxide.

101. The composition of claim 95, wherein the polymer is propyleneglycol, glucose star polymer, silicone polymer, polydimethylsiloxane, polyethylene glycol, carboxymethylcellulose, poly(glycolic acid), poly(lactic-co-glycolic acid), polylactic acid, polycaprolactone (PCL), polyvinylpyrrolidone (PVP), ficoll, dextran, or a combination thereof.

102. The composition of claim 95, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, an ionic surfactant, or a combination thereof.

103. The composition of claim 95, wherein the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, or a combination thereof.

104. The composition of claim 95, wherein the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

105. The composition of claim 95, wherein the antiseptic is phenol, m-cresol, benzyl alcohol, 2-phenyloxyethanol, chlorobutanol, neomycin, benzethonium chloride, gluteraldehyde, or beta-propiolactone.

106. The composition of claim 95, wherein the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

107. The composition of claim 95, wherein the antioxidant is glutathione, ascorbic acid, cysteine, N-acety-L-tryptophanate, tocopherol, histidine or methionine.

108. The composition of claim 95, wherein the organic solvent is dimethyl sulfoxide or N-methyl-2-pyrrolidone.

109. The composition of claim 95, wherein the preservative is methyl hydroxybenzoate, thimerosal, a paraben, formaldehyde, or castor oil.

110. The composition of claim 95, wherein the paraben is a parahydroxybenzoate.

111. The composition of claim 95, wherein the bactericide is benzalkonium chloride or benzyl benzoate.

112. The composition of claim 95, wherein the analgesic is acetaminophen or lidocaine.

113. The composition of any one of claims 45-112, wherein the liquid further comprises at least one pharmaceutically acceptable additive, diluent, excipient, carrier, or a combination thereof.

114. The composition of any one of claims 45-113, wherein the composition has a viscosity of less than about 200 mPa·s.

115. The composition of claim 114, wherein the composition has a viscosity of less than about 150 mPa·s.

116. The composition of claim 114 or 115, wherein the composition has a viscosity of less than about 50 mPa·s.

117. The composition of any one of claims 114-116, wherein the composition has a viscosity of less than about 30 mPa·s.

118. The composition of any one of claims 114-117, wherein the composition has a viscosity of less than about 20 mPa·s.

119. The composition of any one of claims 114-118, wherein the composition has a viscosity of less than about 10 mPa·s.

120. The composition of any one of claims 114-119, wherein the composition has a viscosity of less than about 5 mPa·s.

121. The composition of any one of claims 114-120, wherein the composition has a viscosity of less than about 3 mPa·s.

122. The composition of any one of claims 114-121, wherein the composition has a viscosity of less than about 2.5 mPa·s.

123. The composition of any one of claims 45-122, wherein the composition has improved stability of the peptide compared to an aqueous composition comprising the peptide in monomeric form.

124. A method of forming particles, the method comprising:
a) providing droplets comprising a first liquid and a peptide;
b) contacting the droplets comprising the peptide with a second liquid;
c) allowing the droplets to dry; and
d) removing the first and second liquids,
thereby forming particles comprising a peptide, wherein the particles comprise up to about 10% (w/w) of peptide and the circularity of the particles is from about 0.10 to about 1.00 after removing the first and second liquids.

125. The method of claim 124, wherein the peptide is a biologically active peptide.

126. The method of claim 125, wherein the biologically active peptide comprises from about 2 to about 12 amino acid residues.

127. The method of any one of claims 124-126, wherein the particles comprise up to about 8% (w/w) of peptide.

128. The method of claim 127, wherein the particles comprise up to about 6% (w/w) of peptide.

129. The method of claim 127 or 128, wherein the particles comprise up to about 5% (w/w) of peptide.

130. The method of any one of claims 127-129, wherein the particles comprise up to about 3% (w/w) of peptide.

131. The method of any one of claims 124-130, wherein the circularity of the particles is from about 0.80 to about 1.00 after removing the first and second liquids.

132. The method of claim 131, wherein the circularity of the particles is from about 0.85 to about 1.00 after removing the first and second liquids.

133. The method of claim 131 or 132, wherein the circularity of the particles is from about 0.90 to about 1.00 after removing the first and second liquids.

134. The method of any one of claims 131-133, wherein the circularity of the particles is from about 0.95 to about 1.00 after removing the first and second liquids.

135. The method of any one of claims 131-134, wherein the circularity of the particles is from about 0.98 to about 1.00 after removing the first and second liquids.

136. The method of any one of claims 131-135, wherein the circularity of the particles is about 1.00 after removing the first and second liquids.

137. The method of any one of claims 124-136, wherein the particles have a substantially smooth surface after removing the first and second liquids.

138. The method of any one of claims 124-137, wherein the particles have a diameter of about 0.1 to about 100 μm after removing the first and second liquids.

139. The method of claim 138, wherein the particles have a diameter of about 1 to about 100 μm after removing the first and second liquids.

140. The method of claim 138 or 139, wherein the particles have a diameter of about 5 to about 100 μm after removing the first and second liquids.

141. The method of any one of claims 138-140, wherein the particles have a diameter of about 5 to about 50 μm after removing the first and second liquids.

142. The method of any one of claims 138-141, wherein the particles have a diameter of about 5 to about 20 μm after removing the first and second liquids.

143. The method of any one of claims 124-142, wherein the particles have a skeletal density of about 1.00 to about 6.00 g/cm³ after removing the first and second liquids.

144. The method of claim 143, wherein the particles have a skeletal density of about 1.15 to about 1.60 g/cm³ after removing the first and second liquids.

145. The method of claim 143 or 144, wherein the particles have a skeletal density of about 1.25 to about 1.50 g/cm³ after removing the first and second liquids.

146. The method of any one of claims 143-145, wherein the particles have a skeletal density of about 1.30 to about 1.40 g/cm³ after removing the first and second liquids.

147. The method of any one of claims 124-146, wherein the particles have a glass transition temperature that is higher than about 60° C. after removing the first and second liquids.

148. The method of claim 147, wherein the particles have a glass transition temperature that is higher than about 90° C. after removing the first and second liquids.

149. The method of claim 147 or 148, wherein the particles have a glass transition temperature that is higher than about 100° C. after removing the first and second liquids.

150. The method of any one of claims 147-149, wherein the particles have a glass transition temperature that is higher than about 130° C. after removing the first and second liquids.

151. The method of any one of claims 147-150, wherein the particles have a glass transition temperature that is higher than about 170° C. after removing the first and second liquids.

152. The method of any one of claims 124-151, wherein the particles further comprise a carbohydrate, a protein stabilizer, an emulsifier, an amino acid, a surfactant, or a combination thereof.

153. The method of claim 152, wherein the carbohydrate is dextran, trehalose, sucrose, agarose, mannitol, lactose, sorbitol, maltose, or a combination thereof.

154. The method of claim 152, wherein the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, or a combination thereof.

155. The method of claim 152, wherein the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

156. The method of claim 152, wherein the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

157. The method of claim 152, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof.

158. The method of any one of claims 124-157, wherein the particles have a surfactant content of less than about 10% by mass remaining after removing the first and second liquids.

159. The method of claim 158, wherein the particles have a surfactant content of less than about 5% by mass remaining after removing the first and second liquids.

160. The method of claim 158 or 159, wherein the particles have a surfactant content of less than about 3% by mass remaining after removing the first and second liquids.

161. The method of any one of claims 158-160, wherein the particles have a surfactant content of less than about 1% by mass remaining after removing the first and second liquids.

162. The method of any one of claims 158-161, wherein the particles have a surfactant content of less than about 0.1% by mass remaining after removing the first and second liquids.

163. The method of any one of claims 158-162, wherein the particles are substantially free from any surfactant content after removing the first and second liquids.

164. The method of any one of claims 124-163, wherein the particles have less than about 7% of residual first and second liquid by mass remaining after removing the first and second liquids.

165. The method of claim 164, wherein the particles have less than about 5% of residual first and second liquid by mass remaining after removing the first and second liquids.

166. The method of claim 164 or 165, wherein the particles have less than about 3% of residual first and second liquid by mass remaining after removing the first and second liquids.

167. The method of any one of claims 164-166, wherein the particles have less than about 1% of residual first and second liquid by mass remaining after removing the first and second liquids.

168. The method of any one of claims 124-167, wherein the particles have less than about 7% residual moisture by weight after removing the first and second liquids.

169. The method of claim 168, wherein the particles have less than about 5% residual moisture by weight after removing the first and second liquids.

170. The method of claim 168 or 169, wherein the particles have less than about 3% residual moisture by weight after removing the first and second liquids.

171. The method of any one of claims 168-170, wherein the particles have less than about 1% residual moisture by weight after removing the first and second liquids.

172. The method of any one of claims 124-171, wherein the first liquid is an aqueous liquid, an organic solvent, an ionic liquid, a hydrogel, an ionogel, or a combination thereof.

173. The method of clam 172, wherein the first liquid is an aqueous liquid.

174. The method of claim 172 or 173, wherein the first liquid is water, 0.9% saline, lactated Ringer's solution, a buffer, dextrose 5%, or a combination thereof.

175. The method of any one of claims 172-174, wherein the first liquid is water.

176. The method of clam 174, wherein the buffer is acetate buffer, histidine buffer, succinate buffer, HEPES buffer, tris buffer, carbonate buffer, citrate buffer, phosphate buffer, glycine buffer, barbital buffer, or cacodylate buffer.

177. The method of any one of claims 124-176, wherein the concentration of the peptide in the first liquid is from about 0.0001 mg/mL to about 1000 mg/mL.

178. The method of claim 177, wherein the concentration of the peptide in the first liquid is from about 10 mg/mL to about 500 mg/mL.

179. The method of claim 177 or 178, wherein the concentration of the peptide in the first liquid is from about 10 mg/mL to about 100 mg/mL.

180. The method of any one of claims 177-179, wherein the concentration of the peptide in the first liquid is from about 3 mg/mL to about 25 mg/mL.

181. The method of any one of claims 124-180, wherein the first liquid further comprises a carbohydrate, a protein stabilizer, an emulsifier, an amino acid, a surfactant, or a combination thereof.

182. The method of claim 181, wherein the carbohydrate is dextran, trehalose, sucrose, agarose, mannitol, lactose, sorbitol, maltose, or a combination thereof.

183. The method of claim 181, wherein the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, or a combination thereof.

184. The method of claim 181, wherein the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

185. The method of claim 181, wherein the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof.

186. The method of claim 181, wherein the surfactant is polysorbate, magnesium stearate, sodium dodecyl sulfate, TRITON™ N-101, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, or a combination thereof.

187. The method of any one of claims 124-186, wherein the first liquid has a viscosity from about 0.01 to about 10,000 mPa·s.

188. The method of claim 187, wherein the first liquid has a viscosity of less than about 100 mPa·s.

189. The method of claim 187 or 188, wherein the first liquid has a viscosity of less than about 10 mPa·s.

190. The method of any one of claims 187-189, wherein the first liquid has a viscosity of less than about 3 mPa·s.

191. The method of any one of claims 187-190, wherein the first liquid has a viscosity of less than about 0.9 mPa·s.

192. The method of any one of claims 124-191, wherein the second liquid is an aqueous liquid, an organic solvent, an ionic liquid, a hydrogel, ionogel, protein stabilizer, or a combination thereof.

193. The method of claim 192, wherein the second liquid is an organic solvent.

194. The method of claim 193, wherein the organic solvent is benzyl alcohol, benzyl benzoate, castor oil, coconut oil, corn oil, cottonseed oil, fish oil, grape seed oil, hazelnut oil, hydrogenated palm seed oil, olive oil, peanut oil, peppermint oil, safflower oil, sesame oil, soybean oil, sunflower oil, vegetable oil, walnut oil, polyethylene glycol, glycofurol, acetone, diglyme, dimethylacetamide, dimethyl isosorbide, dimethyl sulfoxide, ethanol, ethyl acetate, butyl acetate, ethyl ether, ethyl lactate, isopropyl acetate, methyl acetate, methyl isobutyl ketone, methyl tert-butyl ether, N-methyl pyrrolidone, perfluorodecalin, 2-pyrrolidone, trigylcerides, tetrahydrofurfuryl alcohol, triglycerides of the fractionated plant fatty acids C8 and C10 (e.g., MIGLYOL® 810 and MIGLOYL® 812N), propylene glycol diesters of saturated plant fatty acids C8 and C10 (e.g., MIGLYOL® 840), ethyl oleate, ethyl caprate, dibutyl adipate, fatty acid esters, hexanoic acid, octanoic acid, triacetin, diethyl glycol monoether, gamma-butyrolactone, eugenol, clove bud oil, citral, limonene, or a combination thereof.

195. The method of claim 193 or 194, wherein the organic solvent is ethyl acetate or butyl acetate.

196. The method of any one of claims 124-195, wherein the second liquid has a viscosity from about 0.01 to about 10,000 mPa·s.

197. The method of claim 196, wherein the second liquid has a viscosity of less than about 10 mPa·s.

198. The method of claim 196 or 197, wherein the second liquid has a viscosity of less than about 5 mPa·s.

199. The method of any one of claims 196-198, wherein the second liquid has a viscosity of less than about 2 mPa·s.

200. The method of any one of claims 196-199, wherein the second liquid has a viscosity of less than about 0.70 mPa·s.

201. The method of any one of claims 196-200, wherein the second liquid has a viscosity of less than about 0.40 mPa·s.

202. The method of any one of claims 124-201, wherein the droplets of step a) are formed by electrospray, an ultrasonic atomizer, vortex, or a microfluidic device.

203. The method of claim 202, wherein the droplets of step a) are formed by an ultrasonic atomizer or by vortex.

204. The method of any one of claims 124-203, wherein the droplets are dried after contacting the droplets comprising the peptide with a second liquid.

205. The method of any one of claims 124-204, wherein step b) further comprises decreasing the temperature of the second liquid to a temperature within about 30° C. of the freezing point of the first liquid.

206. The method of any one of claims 124-205, wherein the boiling point of the second liquid at atmospheric pressure is from about 0 to about 200° C.

207. The method of any one of claims 124-206, wherein the first and second liquids are removed through centrifugation, sieving, filtration, magnetic collection, solvent exchange, or decanting.

208. The method of any one of claims 124-207, further comprising washing the particles after step d) with a washing fluid.

209. The method of claim 208, wherein the washing fluid is an organic liquid, a supercritical fluid, a cryogenic liquid, or a combination thereof.

210. The method of any one of claims 124-209, wherein the particles are further dried by lyophilization or vacuum desiccation.

211. The method of claim 210, wherein the particles are further dried by contacting the particles with a stream of gas.

212. The method of claim 211, wherein the gas has a temperature from about −80 to about 200° C.

213. The method of claim 211 or 212, wherein the gas has a temperature from about −80 to about 200° C.

214. The method of any one of claims 211-213, wherein the gas has a temperature from about 10 to about 40° C.

215. The method of any one of claims 211-214, wherein the gas has a relative humidity greater than about 0% to less than about 100%.

216. The method of any one of claims 211-215, wherein the gas comprises helium, air, nitrogen or argon.

217. The method of any one of claims 124-216, further comprises sterilization of the particles after the first and second liquids are removed.

218. The method of claim 217, wherein the sterilization occurs by irradiation, pasteurization, or freezing.

219. The method of claim 218, wherein the irradiation is by gamma radiation.
